(19) United States
(12) Patent Application Publication
(10) Pub. No.: US 2022/0056075 A1
(43) Pub. Date: Feb. 24, 2022

(54) METHOD FOR TREATING OPIOID USE DISORDER

(71) Applicants: THE ADMINISTRATORS OF THE TULANE EDUCATIONAL FUND, New Orleans, LA (US); DEPARTMENT OF VETERANS AFFAIRS (US), Washington, DC (US)

(72) Inventor: James E. ZADINA, Metaire, LA (US)

(73) Assignees: THE ADMINISTRATORS OF THE TULANE EDUCATIONAL FUND, New Orleans, LA (US); DEPARTMENT OF VETERANS AFFAIRS (US), Washington, DC (US)

(21) Appl. No.: 17/518,047

(22) Filed: Nov. 3, 2021

Related U.S. Application Data
(63) Continuation-in-part of application No. PCT/US2020/031140, filed on May 1, 2020.
(60) Provisional application No. 62/842,954, filed on May 3, 2019.

(51) Int. Cl. C07K 7/06; C07K 7/54

(52) U.S. Cl. CPC C07K 7/06; A61K 38/00; C07K 7/54

(57) ABSTRACT

A method for treating opioid use disorder comprises administering to a subject a pharmaceutical composition comprising a cyclic peptide of Formula I or a pharmaceutically acceptable salt thereof in a pharmaceutically acceptable carrier; wherein the peptide of formula X¹-c[X²-X³-Phe-X⁴]-X⁵ is administered in place of, and as a substitute for an opioid to which the subject is addicted. X¹ is Tyr or 2,6-Dmt; X² is an acidic or basic D-amino acid; X³ is Trp or Phe; there is an amide bond between the sidechains of X² and X⁴; X⁵ is NHR (R=H or alkyl) or an amino acid amide. When X² is an acidic D-amino acid, X⁴ is a basic amino acid, X³ is Phe, and X⁵ is NHR; and when X² is a basic D-amino acid, X⁴ is an acidic amino acid, and X³ is Trp.

### CROSS-REFERENCE TO RELATED APPLICATIONS

This application is a continuation-in-part of PCT/US2020/031140, filed on May 1, 2020, which claims the benefit of U.S. Provisional Application Ser. No. 62/842,954, filed on May 3, 2019, each of which is incorporated herein by reference in its entirety.

### STATEMENT OF GOVERNMENT SUPPORT

A portion of the work described herein was supported by a Merit Review Award, Grant No. I01BX003776, from the Department of Veteran Affairs; Grant No. DM090595 from the Department of Defense; and Grant No. N00014-09-1-0648 from the Office of Naval Research of the Department of Defense. The United States government has certain rights in this invention.

### FIELD OF THE INVENTION

The present invention relates to cyclic peptide agonists that bind to the mu (morphine) opioid receptor and their use in the treatment of opioid use disorder.

### BACKGROUND

Opioid abuse and dependence are widespread problems that cause devastating health consequences. Opioid overdose deaths have more than doubled over the past 10 years due, in part, to co-abuse of prescription opioids, heroin, and/or fentanyl (NIDA, 2017). Opioid use disorders (OUD) are routinely treated with a full mu-opioid receptor (MOR) agonist such as methadone, or a partial agonist such as buprenorphine, for substitution therapy. However, these compounds are tightly regulated because they have their own propensity for abuse indicated by robust intravenous self-administration (SA) rates, locomotor sensitization, and conditioned place preference (CPP) behaviors in rats (Steinpreis et al., 1996; Tzschentke, 2004; Martin et al., 2007; Wade et al., 2015). In humans, buprenorphine and methadone have clinical utility for reducing the positive subjective effects of opioids, but both of these compounds are self-administered and produce positive reinforcing effects (comer et al., 2005; Jones et al., 2014). Novel substitution therapies with low abuse liability compounds may improve treatment for OUD.

A desirable combination of properties for OUD treatment would be a compound that does not induce multiple indices of abuse liability (including CPP, self-administration, or locomotor sensitization) or reduce the size of VTA DA neurons, but which does penetrate the blood brain barrier (BBB) and provide discriminative stimulus effects that are similar to an illicit opioid such as morphine. An important property of a candidate compound for substitution therapy is that the patient experience the compound as somewhat similar to the abused agonist. Typically, a partial agonist meets these requirements while producing fewer reinforcing effects. Although DD is typically used to indicate that the test drug may be abused if subsequent CPP or SA tests confirm abuse liability (Swedberg, 2016), DD may also indicate that the test drug is similar to the training drug, but dissociable from the reward properties of the training drug (Ator, 2002). That is the case with the nicotine replacement therapy, varenicline (Bordia et al., 2012), the active ingredient in Chantix.

Methadone and buprenorphine have played a valuable role in the treatment of OUD, producing effective opioid substitution effects with relatively long durations of action that can reduce the need for subsequent doses. These compounds do, however, retain reward properties and other adverse side-affects. Novel therapies with reduced reward properties could therefore increase the armamentarium of options for treatment and management of OUD.

There is an ongoing need for new treatments for OUD. The methods described herein address this need.

### SUMMARY

Endomorphins (EM) are endogenous tetrapeptides that are highly selective for the MOR (Zadina et al., 1997), the primary analgesic target for opium-derived medications such as morphine. In 2016, Zadina et al. described cyclized D-amino acid-containing EM analogs (Zadina et al., 2016). The suitability of such EM analogs as therapeutics for treating OUD is demonstrated herein using 5 approaches: (1) an extended (5 day) CPP procedure, (2) an examination of locomotor sensitization, a behavior associated with increased dopamine (DA) release (Bohn et al., 2003) and abuse liability (Robinson and Berridge, 2001), (3) examination of BBB penetration and mu-selectivity of ZH853, (4) an assessment of a potential neurobiological reward-tolerance mechanism by which repeated morphine injections reduce the size of ventral tegmental area (VTA) DA neurons (Kish et al., 2001; Chu et al., 2008; Mazei-Robison et al., 2011; Mazei-Robison and Nestler, 2012), and (5) an examination of the interoceptive stimulus effects in a drug discrimination (DD) procedure.

The cyclic EM analog peptides described herein, which are useful as therapeutics for OUD, are high affinity mu opioid receptor agonists of Formula I: H—X¹-cyclo[X²-X³-Phe-X⁴]-X⁵ (which alternatively can be written as X¹-c[X²-X³-Phe-X⁴]-X⁵). X¹ is tyrosine (Tyr) or 2,6-dimethyltyrosine (2,6-Dmt), preferably Tyr. X² is a D-amino acid residue that can be an acidic amino acid (i.e., an amino acid comprising a carboxylic acid-substituted sidechain, such as D-Asp or D-Glu) or basic amino acid (i.e., an amino acid comprising an amino-substituted sidechain, such as D-Lys, D-Orn, D-Dpr, or D-Dab). X³ is Trp or Phe. There is an amide bond between the sidechains of X² and X⁴, such that the substructure X²-X³-Phe-X⁴ constitutes a ring. X⁵ is selected from the group consisting of NHR, Ala-NHR, Arg-NHR, Asn-NHR, Asp-NHR, Cys-NHR, Glu-NHR, Gln-NHR, Gly-NHR, His-NHR, Ile-NHR, Leu-NHR, Lys-NHR, Met-NHR, Orn-NHR, Phe-NHR, Pro-NHR, Ser-NHR, Thr-NHR, Trp-NHR, Tyr-NHR, and Val-NHR; where R is H or an alkyl group (e.g. a (C₁ to C₁₀) alkyl group such as methyl, ethyl, propyl, isopropyl, butyl, isobutyl, pentyl, isopentyl, hexyl, isohexyl, heptyl, or isoheptyl); preferably R is H.

When X² is an acidic D-amino acid, X⁴ is a basic amino acid, X³ is Phe, and X⁵ is NHR (preferably NH₂). When X² is a basic D-amino acid, X⁴ is an acidic amino acid, X³ is Phe, and X⁵ preferably is selected from the group consisting of Ala-NHR, Arg-NHR, Asn-NHR, Asp-NHR, Cys-NHR, Glu-NHR, Gln-NHR, Gly-NHR, His-NHR, Ile-NHR, Leu-NHR, Lys-NHR, Met-NHR, Orn-NHR, Phe-NHR, Pro-NHR, Ser-NHR, Thr-NHR, Trp-NHR, Tyr-NHR, and Val-NHR; where R is H or an alkyl group; preferably R is H.

In some embodiments, the cyclic peptide of Formula I is a hexapeptide of Formula II: Tyr-c[X⁶-Trp-Phe-X⁷]-X⁸-NH₂, wherein X⁶ is selected from the group consisting of D-Lys and D-Orn, and X⁷ is selected from the group consisting of Glu and Asp, and X⁸ is Gly-NH₂ or a conservative substitute for Gly-NH₂.

In some other embodiments, the cyclic peptide of Formula I is a pentapeptide of Formula III: Tyr-c[X⁹-Phe-Phe-X¹⁰]-NH₂, wherein X⁹ is selected from the group consisting of D-Glu and D-Asp, and X¹⁰ is selected from the group consisting of Lys and Orn.

Some preferred peptides for use in the methods described herein are Tyr-c[D-Lys-Trp-Phe-Glu]-Gly-NH₂ (also referred to herein as ZH853), Tyr-c[D-Glu-Phe-Phe-Lys]-NH₂ (also referred to herein as ZH831), and Tyr-c[D-Lys-Trp-Phe-Glu]-NH₂ (also referred to herein as ZH850).

A method for treating opioid use disorder comprises administering to a subject in need thereof pharmaceutical composition comprising a cyclic peptide of Formula I as described herein or a pharmaceutically acceptable salt thereof, in a pharmaceutically acceptable carrier, wherein the peptide is administered in place of, and as a substituted for a mu opioid receptor agonist to which the subject is addicted. In some embodiments the compound of Formula I can be, e.g., a compound of Formula II, such as ZH853. In some other embodiments, the compound of Formula I can be a compound of Formula II, such as ZH831.

In some embodiments, the subject will be addicted to one or more opioid such as, e.g., morphine, oxycodone, hydrocodone, codeine, heroin, oxymorphone, fentanyl, and the like. Often, the subject will have been previously treated for OUD using a drug such as methadone, buprenorphine, naltrexone, suboxone, and the like.

In some embodiments the subject will be treated with intravenously with a cyclic peptide of Formula I, II or III. In other embodiments, the subject will be treated with orally with the peptide of Formula I, II or III. Initial doses of the cyclic peptide may be at a low dose such as a dose that is less than the ED50 for the peptide for analgesia. In some embodiments, the treatment will begin at the low dose and will be increased over time to a higher maintenance level during the course of the treatment.

### BRIEF DESCRIPTION OF THE DRAWINGS

FIG. 1 illustrates conditioned place preference and locomotor effects of morphine and ZH853. After establishing baseline activity, locomotor activity was measured during 5 daily conditioning sessions conducted immediately after injection (i.v.) of morphine, ZH853, or vehicle. (a) Locomotor effects of morphine differed by dose with lower doses producing acute locomotor enhancement, while higher doses (e.g., 3.2 mg/Kg) acutely suppressed locomotion, and then enhanced locomotion after daily administration. (b) Locomotor effects of ZH853 were no different from controls. (c) Subtracting day 1 locomotion from day 5, shows morphine produced locomotor sensitization, while EM ZH853 did not. (d) Conditioned place preference (CPP) effects after 5 days of conditioning shows that ZH853 did not produce CPP or aversive effects, whereas morphine (3.2 mg/Kg i.v.) produced significant CPP. Nearly identical antinociceptive effects of morphine and ZH853 were produced during the same time frame rats underwent CPP conditioning (See (Zadina et al., 2016) for antinociception data). +, ++, +++p<0.05, 0.01, 0.001 compared to vehicle. *, **p<0.05, <0.01 compared to morphine, respectively.

FIG. 2 shows that chronic injections of morphine, but not ZH853, reduced DA cell surface area and volume in the posterior ventral tegmental area (pVTA). (a) Low magnification section of pVTA used for analysis of DA morphology. Rats were perfused after the final CPP test session and pVTA sections were stained with tyrosine hydroxylase (TH). TH+ somas from z-stacks were analyzed by MBF STEREO INVESTIGATOR software for surface area (μm²) and volume (μm³) in the parabrachial pigmented area (PBP) and paranigral area (PN) of the pVTA. (b) An example of PBP somas in which morphine (5.6 mg/Kg, i.v.) reduced the surface area and volume of cell somas, while ZH853 did not alter soma sizes at any dose. (c) Surface area and volume of cell somas were quantified in PBP and PN regions using the STEREO INVESTIGATOR nucleator probe. Scale bars=50 μm (a) or 10 μm (b). 6-8 cell somas were quantified per rat with 5-6 rats per drug group. +p<0.05 compared to vehicle.

FIG. 3 illustrates antinociceptive effects of ZH853 in the hot plate (HP) test. Latencies of mice to lick or shake the paw were measured at regular intervals and converted to % MPE. (a) Values show dose-dependent HP antinociception produced in males by ZH853 that was attenuated by the MOR selective irreversible antagonist βFNA (40 mg/Kg, s.c.). +, ++p<0.05, 0.01 for 5.6 mg/Kg compared to vehicle; ***p<0.001 for 10 mg/Kg compared to vehicle. Values after 10 mg/Kg ZH853+βFNA were not different from vehicle. (b) Area under the curve at various doses for both males and females. ANOVA showed a significant effect of dose F (4, 60)=16.61, p<0.0001, but the effect of sex and the interaction were not significant.

FIG. 4 illustrates morphine discrimination training and substitution testing. (a) Rats were trained to discriminate morphine (3.2 mg/Kg, s.c.) from vehicle (s.c.) and reached criterion after approximately 20 sessions. Rats were then catheterized for i.v. injections, allowed to recover, and continued training to discriminate morphine (1.8 mg/Kg, i.v.) from vehicle (i.v.) injections. The dotted line in indicates the training criteria of 90% drug-appropriate responding. (b) During test sessions in which both levers actively delivered food, rats dose-dependently responded on the drug-paired lever for i.v. morphine after bolus injections made between sessions, or i.v. cumulative injections made within a single session. (c) Response rates for food on the morphine and vehicle levers during morphine discrimination training sessions. Morphine (3.2 mg/Kg s.c. and 1.8 mg/Kg i.v.) produced some impairment of responding during training. (d) Morphine disrupted response rates during test sessions at doses≥3.2 mg/Kg after bolus injections and ≥5.6 mg/Kg after cumulative injections. *, ***, p<0.05, 0.001 compared to vehicle. n=6.

FIG. 5 illustrates discriminative stimulus and response rate effects of EM analogs in morphine-trained rats. Morphine-appropriate lever responding during test sessions in which ZH850 (a), ZH831 (b), or ZH853 (c) were administered with bolus injections made between sessions or cumulative injections made within a single session. The bottom panels show response rates for food (pressings/min) were modestly, but significantly impaired by between-session injection of ZH850, but not after cumulative injections. ZH831 and ZH853 did not impair response rates under either injection method, and fully substituted for morphine. *p<0.05 compared to vehicle. n=6.

FIG. 6 provides a comparison of the pharmacodynamic effects of morphine and ZH853. Morphine antinociception and drug discrimination (DD) dose-response curves (% Response, lefty-axis) and self-administration (SA) intake/h (SA intake mg/Kg, right y-axis) during 12 h SA sessions requiring high FR responding (FR3-5). SA and antinociception data reproduced from Zadina et al. (2016).

FIG. 7 provides a graph depicting the results of a study showing that morphine, but not ZH853, reinstates morphine-induced conditioned place preference. Male Sprague-Dawley rats were exposed to a conditioned place preference (CPP) apparatus for two sessions to determine baseline (BL) preference for 2 distinct, unbiased chambers. They then received morphine and were confined for 45 min in one chamber and vehicle in the other for 4 days, counterbalanced for chamber (equal luminance stripe vs gray), and time of morphine vs vehicle injection (am vs pm). A significant place preference for the morphine chamber was observed (CPP, ** p<0.01 vs BL). The animals then underwent extinction procedures consisting of confinement for 45 min in the previously drug- and vehicle-paired chambers for 7 days. Extinction of morphine place preference was then confirmed by assessing compartment preference during a 20 min session. The following day, rats were given a vehicle priming injection and exposed to the apparatus for 20 min, which did not reinstate CPP. The animals were then divided into two groups that were counterbalanced for baseline and extinction values, and were given equi-antinociceptive priming doses of morphine (1.8 mg/Kg) or ZH853 (1.8 mg/Kg), then monitored for CPP for 20 min. Animals primed with morphine showed reinstatement of CPP (MS prime, **=p<0.01 vs BL), while those primed with ZH853 showed scores not significantly different from baseline.

FIG. 8 provides graphs depicting the results of a study showing that morphine, but not ZH853, produces naloxone-induced conditioned place aversion (CPA). Upper graphs: After 2 days of drug-free preconditioning (Pre-cond) to the entire CPA apparatus, male and female rats received equi-analgesic i.v. doses of morphine (5.6 mg/Kg) or ZH853 (4.2 mg/Kg), or vehicle. Four hours later they received s.c. naloxone (1 mg/Kg) and were confined to one side of the apparatus (counterbalanced) for 20 min. The following day the animals received vehicle-then-saline rather than drug-then-naloxone. This cycle (drug+4 h+nlx+next day vehicle+saline) was repeated once, and the next day the animals had access to the entire apparatus (Post-cond).

FIG. 9 illustrates locomotor effects of morphine and ZH853 in Mice. ZH853 provided equivalent antinociception of similar duration as morphine. Tail flick latency is illustrated in Panels (A) through (D) for s.c. administration of morphine and ZH853 compared to vehicle-treated male (a and c) and female (B and D) mice. Data are presented as percent maximum possible effect (% MPE) (A and B), or AUC (C and D). n=5-7 per group. Acute morphine injection increased the AUCs of distance traveled in male (E and G) and female (F and H) mice. Administration of an equi-antinociceptive dose of ZH853 did not affect the distance traveled compared to vehicle-treated mice. n=7-8 per group. ++, +++, ++++=p<0.01, 0.001, 0.0001 compared to vehicle. **, ***=p<0.01, 0.001 compared to morphine.

FIG. 10 illustrates alleviation of morphine withdrawal signs by ZH853. Rats were pretreated (PreTx) for 5 days with vehicle (Veh) or escalating doses of morphine sulfate (MS), then allowed 24 hr to develop spontaneous withdrawal symptoms. The animals were then challenged with Veh or ZH853 at 1.8 or 3.2 mg/Kg and withdrawal symptoms quantified. (A) Analysis of Variance of the Global Withdrawal (GWD) Scores revealed a significant (p<0.05) effect of treatment, and post-hoc Newman-Kuels tests showed that, when challenged with Veh, animals given MS PreTx (MS-Veh) produced significantly greater withdrawal (p<0.01, ++) than Veh-PreTx+Veh challenge (Veh-Veh). By contrast, challenge with 853 (1.8 and 3.2 mg/Kg) after morphine PreTx produced significantly lower GWD scores than MS-Veh (p<0.05, *), consistent with blockade of MS-induced withdrawal. (B) Analysis of Wet Dog Shakes (WDS), a component of the GWD score, revealed a significant effect of treatment (p<0.01), and a significant increase in WDS after MS PreTx+Veh challenge relative to Veh-Veh (p<0.01, ++). MS PreTx and challenge with ZH853 produced significantly lower WDS than Veh challenge for both doses of ZH853 (p<0.05, *), consistent with blockade of MS-induced withdrawal (n=7,7,3, and 3 for Veh-Veh, MS-Veh, MS-ZH853/1.8, and MS-ZH853/3.2, respectively).

FIG. 11 illustrates results of oxycodone self-administration tests. Rats acquired intravenous oxycodone self-administration (0.1 mg/kg/infusion sessions 1-5, 0.05 mg/kg/infusion sessions 6-15), indicated by an increase in infusions obtained: (A) active lever responses, and (B) active lever preference, over fifteen 3-hour sessions. Rats that met the criterion of (1)≥15 active lever responses and (2) a ratio active/inactive lever pressings≥1.5 for the last 3 acquisition sessions were included in the maintenance phase of the study. During the maintenance phase, oxycodone (0.05 mg/kg/infusion) remained available during SA sessions or was substituted with equi-antinociceptive ZH853 (0.25 mg/kg/infusion) or vehicle. ZH853 and vehicle substitution reduced the number of infusions obtained (C) and active lever responses (D) over maintenance sessions. N=5-7 per group. †, ††, †††, †††† p<0.05, 0.01, 0.001, 0.0001 compared to session 1, ###, ####p<0.001, 0.0001 compared to the inactive lever, +p<0.05 ZH853 compared to vehicle, *, **, ***, **** p<0.05, 0.01, 0.001, 0.0001 ZH853 compared to oxycodone. §, § §, § § §, § § § § p<0.05, 0.01, 0.001, 0.0001 vehicle compared to oxycodone.

FIG. 12 illustrates results of oxycodone self-administration tests and relapse. Rats acquired oxycodone self-administration (see FIG. 11), indicated by an increase in infusions obtained: (A) active lever responses, and (B) active lever preference over 15 sessions. Rats that met the criterion described in FIG. 11 were subject to 7 days of forced oxycodone abstinence in their home cages. The 1-hour relapse test occurred the 8^{th} day of forced abstinence. Rats were pretreated with 1.8 mg/kg ZH853, 3.2 mg/kg ZH853, 0.32 mg/kg oxycodone, or vehicle (i.v.) and placed in SA chambers under the same condition as during the acquisition phase, but responses on the active lever did not lead to drug infusion. ZH853 pretreatment decreased active lever responding compared to vehicle and oxycodone pretreatment (C, D). n=5-7 per group. †, ††, ††† p<0.05, 0.01, 0.001 compared to session 1, #, ##, ###, ####p<0.05, 0.01, 0.001, 0.0001 compared to the inactive lever, +, ++++p<0.05, 0.0001 ZH853 compared to vehicle, *, ***, **** p<0.05, 0.001, 0.0001 ZH853 compared to oxycodone, § p<0.05 1.8 mg/kg ZH853 compared to 3.2 mg/kg ZH853.

### DETAILED DESCRIPTION

Peptides of Formula I are cyclic pentapeptide and hexapeptide analogs of endomorphin-1 and endomorphin-2 which are useful for treating opioid use disorder. In one embodiment, a method for treating opioid use disorder comprises administering to a subject in need thereof a pharmaceutical composition comprising a cyclic peptide of Formula I or a pharmaceutically acceptable salt thereof in a pharmaceutically acceptable carrier; wherein the peptide of Formula I is administered in place of, and as a substituted for a mu opioid receptor agonist to which the subject is addicted. Formula I is a cyclic peptide of generic formula X¹-c[X²-X³-Phe-X⁴]-X⁵. X¹ is Tyr or 2,6-Dmt; X² is an acidic or basic D-amino acid; X³ is Trp or Phe; there is an amide bond between the sidechains of X² and X⁴; X⁵ is NHR (R=H or alkyl) or an amino acid amide; provided that: when X² is an acidic D-amino acid, X⁴ is a basic amino acid, X³ is Phe, and X⁵ is NHR; and when X² is a basic D-amino acid, X⁴ is an acidic amino acid, and X³ is Trp. Some examples of peptides in which X³ is Trp include Tyr-c[D-Lys-Trp-Phe-Glu]-Gly-NH₂ (also referred to as ZH853), and Tyr-c[D-Lys-Trp-Phe-Glu]-NH₂ (also referred to as ZH850). In some embodiments, X¹ preferably is Tyr.

In some preferred embodiments, the compound of Formula I is a is hexapeptide of formula Tyr-c[X⁶-Trp-Phe-X⁷]-X⁸—NH₂ (Formula II), wherein X⁶ is selected from the group consisting of D-Lys and D-Orn, and X⁷ is selected from the group consisting of Glu and Asp, and X⁸ is Gly-NH₂ or a conservative substitute for Gly-NH₂. Some examples of conservative substitutes for Gly-NH₂ include Ala-NH₂, Ser-NH₂, and Asn-NH₂. One preferred example of a peptide of Formula II is Tyr-c[D-Lys-Trp-Phe-Glu]-Gly-NH₂ (ZH853).

In some other preferred embodiments, the cyclic peptide of Formula I is a pentapeptide of formula Tyr-c[X⁹-Phe-Phe-X¹⁰]-NH₂ (Formula III), wherein X⁹ is selected from the group consisting of D-Glu and D-Asp, and X¹⁰ is selected from the group consisting of Lys and Orn. One preferred peptide of Formula III is Tyr-c[D-Glu-Phe-Phe-Lys]-NH₂ (ZH831).

In some embodiments of Formula I, X² is a basic D-amino acid; X⁴ is an acidic amino acid; X³ is Trp; and X⁵ is selected from the group consisting of Ala-NHR, Arg-NHR, Asn-NHR, Asp-NHR, Cys-NHR, Glu-NHR, Gln-NHR, Gly-NHR, His-NHR, Ile-NHR, Leu-NHR, Lys-NHR, Met-NHR, Orn-NHR, Phe-NHR, Pro-NHR, Ser-NHR, Thr-NHR, Trp-NHR, Tyr-NHR, and Val-NHR.

In another embodiment of Formula I, X² is an acidic D-amino acid, X⁴ is a basic amino acid, X³ is Phe, and X⁵ is NH₂.

In yet another embodiment of Formula I, X² is selected from the group consisting of D-Asp and D-Glu; X⁴ is selected from the group consisting of Lys, Orn, Dpr and Dab; and preferably, X⁵ is Gly-NH₂.

In another embodiment of Formula I, X² is selected from the group consisting of D-Lys, D-Orn, D-Dpr and D-Dab; X⁴ is selected from the group consisting of Asp and Glu; and preferably, X⁵ is NH₂.

Typically, the dose of the peptide of Formula I will vary over the course of treatment. For example, the treatment may begin at a selected dose, and may be increased over the course of treatment based on the response of the patient to the peptide. In some embodiments, the initial dose of the peptide will be a dose that is less than the analgesic ED50 for a human subject, which may be increased over time to a higher maintenance dose.

In the methods described herein, the subject being treated can be addicted to any mu opioid receptor agonist or a combination of mu-opioid receptor agonists. For example, the subject may be addicted to a mu-opioid receptor agonist such as morphine, oxycodone, hydrocodone, codeine, heroin, oxymorphone, fentanyl, or some combination to two or more such materials. In some cases, the subject may have been treated for OUD with another drug, such as methadone, buprenorphine, naltrexone, suboxone, and the like.

According to the U.S. Department of Health and Human Services National Institute on Drug Abuse (NIDA), addiction is defined as a chronic, relapsing disorder characterized by compulsive drug seeking, continued use despite harmful consequences, and long-lasting changes in the brain. It is considered both a complex brain disorder and a mental illness. Addiction is the most severe form of a full spectrum of substance use disorders, and is a medical illness caused by repeated misuse of a substance or substances. See NIDA Media Guide, printed October 2016, revised July 2018, available at the website drugabuse(dot)gov.

### Methods of Preparation of the Peptides of Formula I.

The peptides of Formula I can be prepared by conventional solution phase or solid phase methods with the use of proper protecting groups and coupling agents. For example, cyclic peptides of Formula I can be synthesized on Rink Amide resin via Fmoc chemistry. A t-butyl group was used for Tyr, Glu, Asp side chain protection and Boc was used for Lys, Orn and Trp side chain protection. The peptide is assembled on Rink Amide resin by repetitive removal of the Fmoc protecting group and coupling of protected amino acid. HBTU (O-benzotriazole-N,N,N′,N′-tetramethyluronium hexafluorophosphate; CAS #94790-37-1) and HOBT (N-hydroxybenzotriazole; CAS #2592-95-2) are used as coupling reagents in N,N-dimethylformamide (DMF), and diisopropylethylamine (DIPEA) is used as a base. The resin is treated with an aqueous cocktail of trifluoroacetic acid and triisopropylsilane (TFA/TIS/H₂O cocktail) for cleavage and removal of the side chain protecting groups. Crude peptide is precipitated with diethyl ether and collected by filtration.

Cyclization of the linear peptide precursors: About 1 mmol of peptide is dissolved in about 1000 mL DMF and about 2 mmol DIPEA is added to the solution, followed by a solution of HBTU (about 1.1 mmol) and HOBT (about 1.1 mmol) in about 100 mL DMF. The reaction mixture is stirred at room temperature overnight. Solvent is removed in vacuo. The resulting solid residue is washed with 5% citric acid, saturated NaCl, saturated NaHCO₃, and water. The final solid is washed with diethyl ether and dried under high vacuum. The solids obtained above are dissolved in 20% piperidine/DMF, and the resulting solution is stirred at room temperature for about 1 hour. Solvent is removed in vacuo. Residues are dissolved in 10% aqueous acetonitrile (MeCN/H₂O) and lyophilized.

Purification of the crude lyophilized peptides is performed with reverse phase high performance liquid chromatography (RP-HPLC). The HPLC system, e.g., a GOLD 32 KARAT (Beckman) system consisting of a programmable solvent module and a diode array detector module is used in the purification and the purity control of the peptides. Reverse phase HPLC is performed using a gradient made from two solvents: (A) 0.1% TFA in water and (B) 0.1% TFA in acetonitrile. For preparative runs, a VYDAC 218TP510 column (250×10 mm; Alltech Associates, Inc.) is used with a gradient of 5-20% solvent B in solvent A over a period of 10 min, 20-25% B over a period of 30 minutes, 25-80% B over a period of 1 minute and isocratic elution over 9 minutes at a flow rate of about 4 mL/min, absorptions being measured at both 214 and 280 nm. The same gradient is used for analytical runs on a VYDAC 218TP54 column (250×4.6 mm) at a flow rate of about 1 mL/min.

Various salt forms of the cyclic peptides can be obtained by acidifying the neutral peptide with an acid to form an acid addition salt, or by anion exchange of one acid addition salt to form another acid addition salt.

### Pharmaceutical Preparations.

The peptides are incorporated in pharmaceutical preparations which contain a pharmaceutically effective amount of the peptide in a pharmaceutically acceptable carrier (e.g., a diluent, complexing agent, additive, excipient, adjuvant and the like). The peptide can be present for example in a salt form, a micro-crystal form, a nano-crystal form, a co-crystal form, a nanoparticle form, a microparticle form, or an amphiphilic form. Salt forms can be, e.g., salts of inorganic acids such as hydrochloride salts, phosphate salts, sulfate salts, bisulfate salts, hemisulfate salts, and the like; or salts of organic acids, such as acetate salts, aspartate salts, citrate salts, fumarate salts, maleate salts, malate salts, lactate salts, hippurate salts, tartrate salts, gluconate salts, succinate salts, and the like. The carrier can be an organic or inorganic carrier, or a combination thereof, which is suitable for external, enteral or parenteral applications. The peptides can be compounded, for example, with the usual non-toxic, pharmaceutically acceptable carriers for tablets, pellets, capsules, liposomes, suppositories, intranasal sprays, solutions, emulsions, suspensions, aerosols, and any other form suitable for use in living subjects, such as human subjects. Non-limiting examples of carriers that can be used include water, glucose, lactose, gum acacia, gelatin, mannitol, starch paste, magnesium trisilicate, talc, corn starch, keratin, colloidal silica, potato starch, urea, glycol ethers, and other carriers suitable for use in manufacturing preparations, in solid, semisolid, and liquid forms. In addition, auxiliary, stabilizing, thickening, flavoring, and coloring agents can be used.

Pharmaceutical compositions useful for treating opioid use disorder utilizing the compounds of Formula I are described herein. The pharmaceutical compositions comprise at least one peptide of Formula I in combination with a pharmaceutically acceptable carrier, vehicle, or diluent, such as an aqueous buffer at a physiologically acceptable pH (e.g., pH 7 to 8.5), a polymer-based nanoparticle vehicle, a liposome, and the like. The pharmaceutical compositions can be delivered in any suitable dosage form, such as a liquid, gel, solid, cream, or paste dosage form. In one embodiment, the compositions can be adapted to give sustained release of the peptide. Aqueous vehicles for the peptides can include a pharmaceutically acceptable cosolvent, e.g., to aid in dissolving the peptides. Non-limiting examples of such cosolvents include, e.g., poly(ethylene glycol) compounds (PEG) such PEG-200, PEG-300, or PEG-400; amide solvents such as dimethylacetamde and N-methyl-2-pyrrolidone; ethanol; propylene glycol; glycerin; and the like.

In some embodiments, the pharmaceutical compositions include, but are not limited to, those forms suitable for oral, topical (including buccal and sublingual), transdermal, parenteral (including intramuscular, subcutaneous, and intravenous), spinal (epidural, intrathecal), and central (intracerebroventricular) administration. The compositions can, where appropriate, be conveniently provided in discrete dosage units. The pharmaceutical compositions can be prepared by any of the methods well known in the pharmaceutical arts. Some preferred modes of administration include intravenous (iv), topical, subcutaneous, oral and spinal.

Pharmaceutical formulations suitable for oral administration include capsules, cachets, or tablets, each containing a predetermined amount of one or more of the peptides, as a powder or granules. In another embodiment, the oral composition is a solution, a suspension, or an emulsion. Alternatively, the peptides can be provided as a bolus, electuary, or paste. Tablets and capsules for oral administration can contain conventional excipients such as binding agents, fillers, lubricants, disintegrants, colorants, flavoring agents, preservatives, or wetting agents. The tablets can be coated according to methods well known in the art, if desired. Oral liquid preparations include, for example, aqueous or oily suspensions, solutions, emulsions, syrups, or elixirs. Alternatively, the compositions can be provided as a dry product for constitution with water or another suitable vehicle before use. Such liquid preparations can contain conventional additives such as suspending agents, emulsifying agents, non-aqueous vehicles (which may include edible oils), preservatives, and the like. The additives, excipients, and the like typically will be included in the compositions for oral administration within a range of concentrations suitable for their intended use or function in the composition, and which are well known in the pharmaceutical formulation art. The peptides are included in the compositions within a therapeutically useful and effective concentration range, as determined by routine methods that are well known in the medical and pharmaceutical arts. For example, a typical composition can include one or more of the peptides at a concentration in the range of at least about 0.01 nanomolar to about 1 molar, preferably at least about 1 nanomolar to about 100 millimolar.

Pharmaceutical compositions for parenteral, spinal, or central administration (e.g. by bolus injection or continuous infusion) can be provided in unit dose form in ampoules, pre-filled syringes, small volume infusion, or in multi-dose containers, and preferably include an added preservative. The compositions for parenteral administration can be suspensions, solutions, or emulsions, and can contain excipients such as suspending agents, stabilizing agents, and dispersing agents. Alternatively, the peptides can be provided in powder form, obtained by aseptic isolation of sterile solid or by lyophilization from solution, for constitution with a suitable vehicle, e.g. sterile, pyrogen-free water, before use. The additives, excipients, and the like typically will be included in the compositions for parenteral administration within a range of concentrations suitable for their intended use or function in the composition, and which are well known in the pharmaceutical formulation art. The peptides are be included in the compositions within a therapeutically useful and effective concentration range, as determined by routine methods that are well known in the medical and pharmaceutical arts. For example, a typical composition can include one or more of the peptides at a concentration in the range of at least about 0.01 nanomolar to about 100 millimolar, preferably at least about 1 nanomolar to about 10 millimolar.

Pharmaceutical compositions for topical administration of the peptides to the epidermis (mucosal or cutaneous surfaces) can be formulated as ointments, creams, lotions, gels, or as a transdermal patch. Such transdermal patches can contain penetration enhancers such as linalool, carvacrol, thymol, citral, menthol, t-anethole, and the like. Ointments and creams can, for example, include an aqueous or oily base with the addition of suitable thickening agents, gelling agents, colorants, and the like. Lotions and creams can include an aqueous or oily base and typically also contain one or more emulsifying agents, stabilizing agents, dispersing agents, suspending agents, thickening agents, coloring agents, and the like. Gels preferably include an aqueous carrier base and include a gelling agent such as cross-linked polyacrylic acid polymer, a derivatized polysaccharide (e.g., carboxymethyl cellulose), and the like. The additives, excipients, and the like typically are included in the compositions for topical administration to the epidermis within a range of concentrations suitable for their intended use or function in the composition, and which are well known in the pharmaceutical formulation art. The peptides are included in the compositions within a therapeutically useful and effective concentration range, as determined by routine methods that are well known in the medical and pharmaceutical arts. For example, a typical composition can include one or more of the peptides at a concentration in the range of at least about 0.01 nanomolar to about 1 molar, preferably at least about 1 nanomolar to about 100 millimolar.

Pharmaceutical compositions suitable for topical administration in the mouth (e.g., buccal or sublingual administration) include lozenges comprising the peptide in a flavored base, such as sucrose, acacia, or tragacanth; pastilles comprising the peptide in an inert base such as gelatin and glycerin or sucrose and acacia; and mouthwashes comprising the active ingredient in a suitable liquid carrier. The pharmaceutical compositions for topical administration in the mouth can include penetration enhancing agents, if desired. The additives, excipients, and the like typically will be included in the compositions of topical oral administration within a range of concentrations suitable for their intended use or function in the composition, and which are well known in the pharmaceutical formulation art. The peptides are included in the compositions within a therapeutically useful and effective concentration range, as determined by routine methods that are well known in the medical and pharmaceutical arts. For example, a typical composition can include one or more of the peptides at a concentration in the range of at least about 0.01 nanomolar to about 1 molar, preferably at least about 1 nanomolar to about 100 millimolar.

Optionally, the pharmaceutical compositions can include one or more other therapeutic agent besides the cyclic peptide of Formula I, e.g., as a combination therapy. The additional therapeutic agent will be included in the compositions within a therapeutically useful and effective concentration range, as determined by routine methods that are well known in the medical and pharmaceutical arts. The concentration of any particular additional therapeutic agent may be in the same range as is typical for use of that agent as a monotherapy, or the concentration may be lower than a typical monotherapy concentration if there is a synergy when combined with a peptide of the present invention.

All the embodiments of the peptides of Formula I can be in the “isolated” state. For example, an “isolated” peptide is one that has been completely or partially purified. In some instances, the isolated compound will be part of a greater composition, buffer system or reagent mix. In other circumstances, the isolated peptide may be purified to homogeneity. A composition may comprise the peptide or compound at a level of at least about 50, 80, 90, or 95% (on a molar basis or weight basis) of all the other species that are also present therein. Mixtures of the peptides of Formula I may be used in practicing methods provided herein.

As used herein, any reference to a peptide of “Formula I” is to be interpreted as also encompassing peptides of Formula II and Formula III. For reference, the abbreviations for amino acids described herein include alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), valine (Val), ornithine (Orn), 2,3-diaminopropionic acid (Dpr), and 2,4-diaminobutyric acid (Dab). The L- or D-enantiomeric forms of these and other amino acids can be included in the peptides of Formula I. Other amino acids, or derivatives or unnatural forms thereof such as those listed in the 2009/2010 Aldrich Handbook of Fine Chemicals (incorporated herein by reference in its entirety, particularly those sections therein listing amino acid derivatives and unnatural amino acids) can be used in preparing compounds of the invention.

The term “analgesic ED50”, as used herein with respect to a peptide of Formula I, refers to a dose of the peptide which provides 50% analgesia as determined for alleviation of intradermal capsaicin-induced pain by the Dixon sequential up-down method (see, e.g., Wong 2014). The analgesic ED50 dose may be different for different methods of administration (e.g., oral, intravenous, intrathecal, subcutaneous, transdermal, and the like), as is well known in the pharmaceutical arts. In rats, a suitable model for determining a dose response curve and an ED50 for antinociception (an animal model for analgesia) is the tail flick test (see Zadina 2016). Based on tail flick studies, the antinociceptive ED50 for ZH853 in rats is about 2 mg/Kg.

As used herein, the terms “reducing,” “inhibiting,” “blocking,” “preventing”, alleviating,” or “relieving” when referring to a peptide, mean that the peptide brings down the occurrence, severity, size, volume, or associated symptoms of a condition, event, or activity by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, or 100% compared to how the condition, event, or activity would normally exist without application of the peptide or a composition comprising the peptide. The terms “increasing,” “elevating,” “enhancing,” “upregulating,” “improving,” or “activating” when referring to a compound mean that the peptide increases the occurrence or activity of a condition, event, or activity by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, 100%, 150%, 200%, 250%, 300%, 400%, 500%, 750%, or 1000% compared to how the condition, event, or activity would normally exist without application of the peptide or a composition comprising the peptide.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans. In some embodiments, the subject is pediatric (e.g., from birth through age 21).

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating”, “alleviating”, “relief” or “treatment” refers to (1) inhibiting the condition (e.g., pain) in an individual who is experiencing or displaying the symptomatology of the condition (i.e., arresting further development of the pathology and/or symptomatology), or (2) ameliorating the condition; for example, ameliorating a condition in an individual who is experiencing or displaying the pathology or symptomatology of the condition (i.e., reversing the pathology and/or symptomatology).

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The terms “consisting of” and “consists of” are to be construed as closed terms, which limit any compositions or methods to the specified components or steps, respectively, that are listed in a given claim or portion of the specification. In addition, and because of its open nature, the term “comprising” broadly encompasses compositions and methods that “consist essentially of” or “consist of” specified components or steps, in addition to compositions and methods that include other components or steps beyond those listed in the given claim or portion of the specification. Recitation of ranges of values herein are merely intended to serve as a shorthand method of referring individually to each separate value falling within the range, unless otherwise indicated herein, and each separate value is incorporated into the specification as if it were individually recited herein. All numerical values obtained by measurement (e.g., weight, concentration, physical dimensions, removal rates, flow rates, and the like) are not to be construed as absolutely precise numbers, and should be considered to encompass values within the known limits of the measurement techniques commonly used in the art, regardless of whether or not the term “about” is explicitly stated. All methods described herein can be performed in any suitable order unless otherwise indicated herein or otherwise clearly contradicted by context. The use of any and all examples, or exemplary language (e.g., “such as”) provided herein, is intended merely to better illuminate certain aspects of the invention and does not pose a limitation on the scope of the invention unless otherwise claimed. No language in the specification should be construed as indicating any non-claimed element as essential to the practice of the invention.

The following examples are included to demonstrate certain features and aspects of the methods and uses described herein. It should be appreciated by those of skill in the art that the techniques disclosed in the examples, which represent techniques known to function well in practicing the methods, can be considered to constitute preferred modes for its practice. However, those of skill in the art should, in light of the present disclosure, appreciate that many changes can be made in the specific disclosed embodiments and still obtain a like or similar result without departing from the spirit and scope of the methods described herein. The examples are provided for illustration purposes only and are not intended to be limiting.

### EXAMPLES

The studies described herein assess the suitability of ZH853 for treatment of opioid use disorders using five approaches: (1) an extended (5 day) CPP procedure; (2) an examination of locomotor sensitization, a behavior associated with increased dopamine (DA) release (Bohn et al., 2003) and abuse liability (Robinson and Berridge, 2001); (3) examination of BBB penetration and mu-selectivity of ZH853; (4) an assessment of a potential neurobiological reward-tolerance mechanism by which repeated morphine injections reduce the size of ventral tegmental area (VTA) DA neurons (Kish et al., 2001; Chu et al., 2008; Mazei-Robison et al., 2011; Mazei-Robison and Nestler, 2012); and (5) an examination of the interoceptive stimulus effects of ZH831, ZH850, and ZH853 in a drug discrimination (DD) procedure.

### Example 1: Locomotor Sensitization

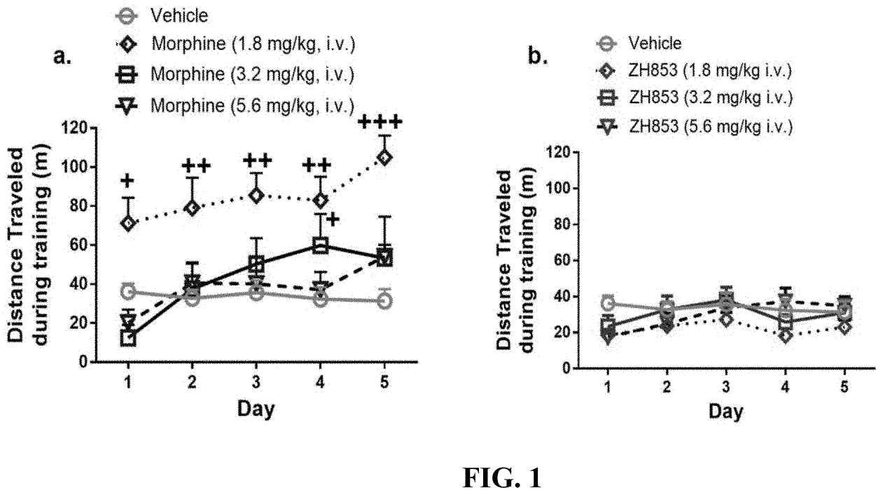

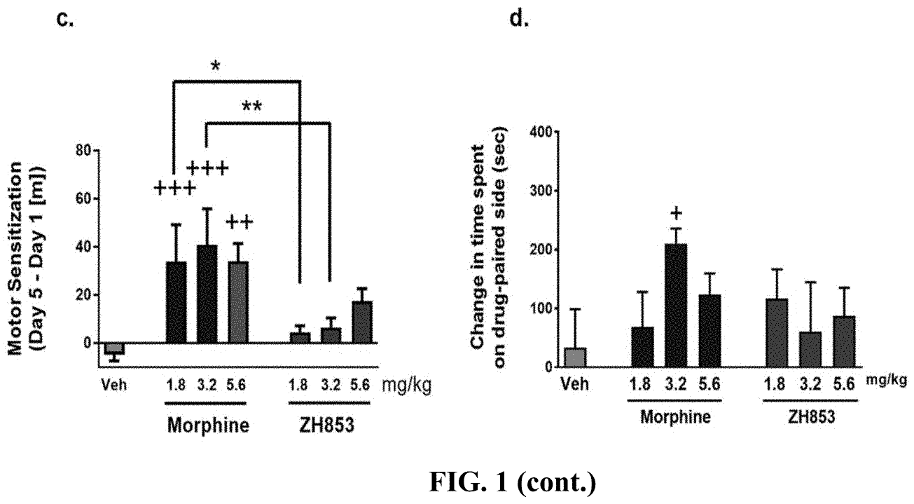

Daily morphine injections produced locomotor sensitization (LS) as measured by an increased distance traveled relative to controls as shown in FIG. 1, Panel a [treatment effect: F (_{3, 28})=7.493, p=0.0008; day effect: F (_{4, 112})=13.16, p<0.0001; interaction: F (_{12, 112})=4.131, p<0.0001]. The 1.8 mg/Kg dose of morphine increased locomotion across all sessions, whereas higher doses (3.2 and 5.6 mg/Kg) initially suppressed locomotion, followed by a gradual increase that indicated LS (Robinson and Berridge, 2001). ZH853 did not produce LS at any dose tested, as shown in FIG. 1, Panel b [F (_{4, 36})=1.6, p=0.1956, n.s.]. When comparing the difference between day 5 and day 1, morphine [F (_{6, 47})=7.635, p<0.0001], but not ZH853, produced significant LS. Post-hoc comparisons showed that compared to vehicle, morphine produced LS at all doses tested [1.8 and 3.2 mg/Kg, p<0.001, 5.6 mg/Kg, p<0.01], while ZH853 did not produce LS at any dose tested (p<n.s.), as shown in FIG. 1, Panel c. Morphine produced conditioned place preference (CPP) effects after 5 days of conditioning [F (_{3, 25})=4.173, p=0.0159] with the 3.2 mg/Kg dose [p<0.05], as shown in FIG. 1, Panel d. The 1.8 and 5.6 mg/Kg doses of morphine did not produce significant CPP consistent with our 3-day injection model (Zadina et al., 2016). ZH853 did not produce CPP (or aversion) at any dose, as shown in FIG. 1D. [F (_{3, 29})=0.9523, p=0.4283 n.s.].

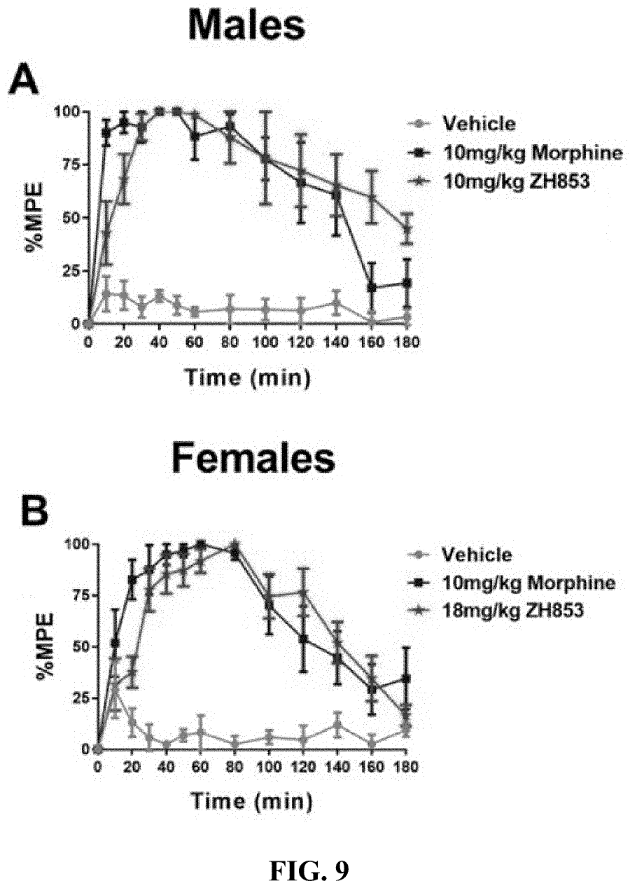

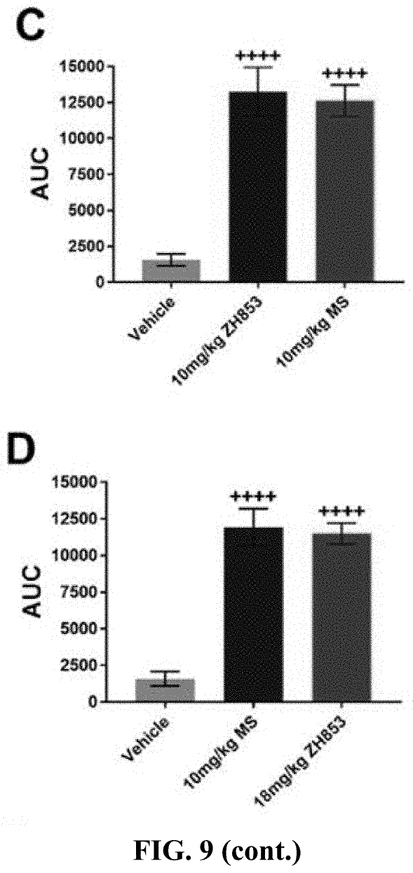

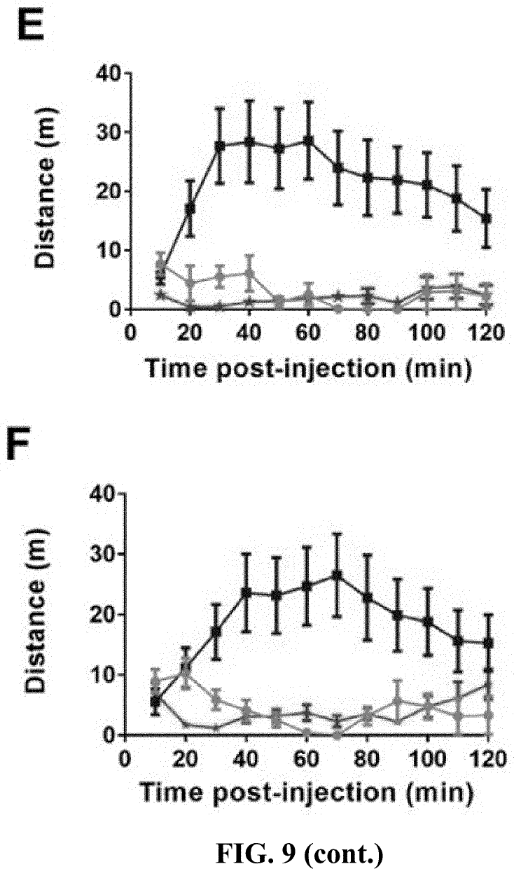

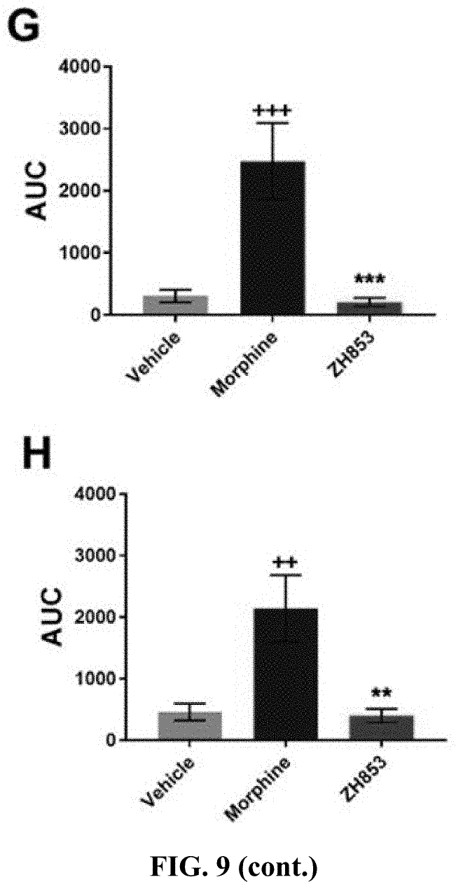

Similar locomotor experiments were performed on male and female mice. FIG. 9 shows that ZH853 provided equivalent antinociception of similar duration as was provided by morphine. S.c. administration of morphine and ZH853 increased the latency to tail flick compared to vehicle-treated male (a and c) and female (B and D) mice. Data are presented as percent maximum possible effect (% MPE) (A and B), or AUC (C and D). n=5-7 per group. Acute administration of morphine, but not equi-antinociceptive doses of ZH853, induced locomotor activation. Basal locomotor activity prior to drug administration did not differ among experimental groups (data not shown). Acute morphine injection increased the AUCs of distance traveled in male (E and G) and female (F and G) mice. Administration of an equi-antinociceptive dose of ZH853 did not affect the distance traveled compared to vehicle-treated mice. n=7-8 per group. ++, +++, ++++=p<0.01, 0.001, 0.0001 compared to vehicle. **, ***=p<0.01, 0.001 compared to morphine.

### Example 2: VTA Dopamine Cell Soma Morphology

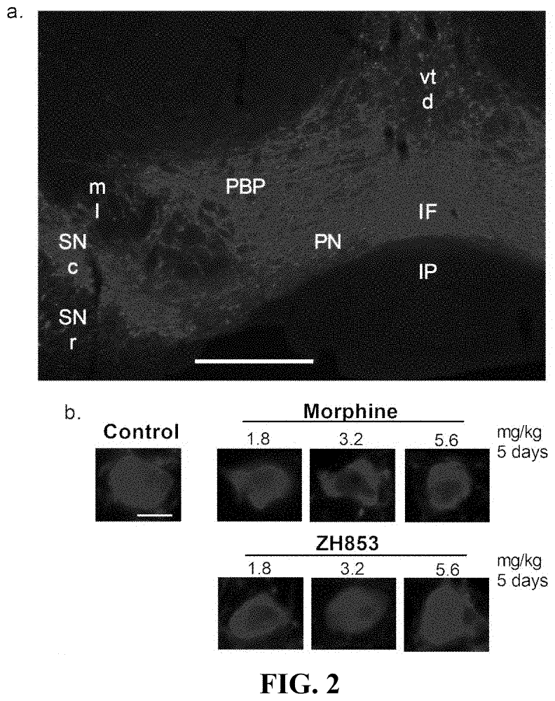

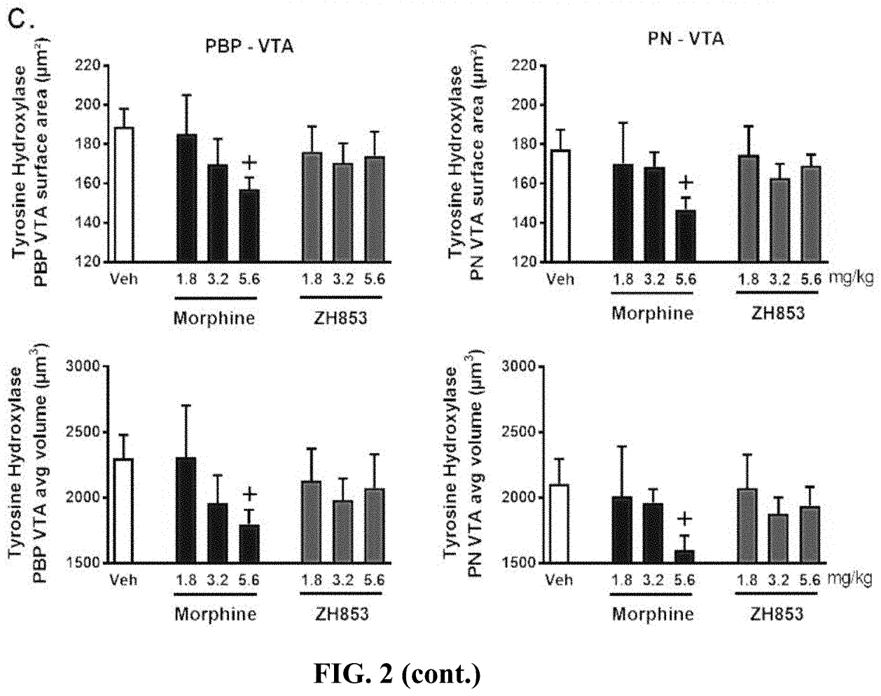

Rats injected with morphine for 5 consecutive days showed a dose-dependent reduction in size of dopamine (DA) neurons in the posterior VTA (FIG. 2, Panel a). Surface area [F (_{2, 27})=6.096, p=0.0065, FIG. 2, Panel b and volume [F (_{2, 27})=4.185, p=0.0261] of tyrosine hydroxylase (TH)-positive somas were reduced by morphine (5.6 mg/Kg, p<0.05). By contrast, ZH853 did not alter either surface area [F (_{3, 32})=0.9463, p=n.s.] or volume [F (_{3, 32})=0.9590, p=n.s.] of DA neurons in the pVTA as measured by STEREO INVESTIGATOR software. Thus, daily injections of antinociceptive doses of morphine, but not ZH853, altered DA soma sizes in the pVTA.

### Example 3: Hot Plate Antinociception after ZH853 and Reversal by the Selective MOR Antagonist βFNA

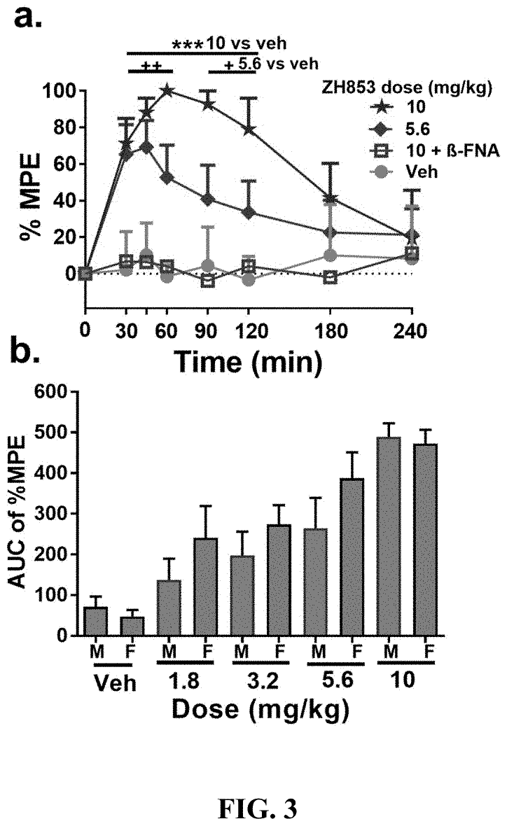

ZH853 dose-dependently increased reaction latencies on the HP test in male DBA mice with maximal antinociception at a dose of 10 mg/Kg (FIG. 3, Panel a). ANOVA showed significant effects of dose [F (_{2, 136})=40.4, p<0.0001], time [F (7,136)=6.7, p<0.0001] and interaction [F (_{14, 136})=2.5 p<0.01]. Significant increases in latencies lasting up to 2 hours were observed for both 5.6 and 10 mg/Kg. βFNA (40 mg/Kg, s.c.) was found to have no antinociceptive properties alone, however 24 h pretreatment with βFNA (40 mg/Kg, s.c.) reversed the antinociceptive effects of the high dose of ZH853 (p<0.001). ZH853 showed similar dose-dependent efficacy in female DBA mice. FIG. 3, Panel b compares the area under the curve at various doses for both males and females. ANOVA showed a significant effect of dose F (_{4, 60})=16.61, p<0.0001, but the effects of sex and the interaction were not significant.

### Example 4: ZH853 Substituted for Morphine with Less Disruption in the Drug Discrimination (DD) Procedure

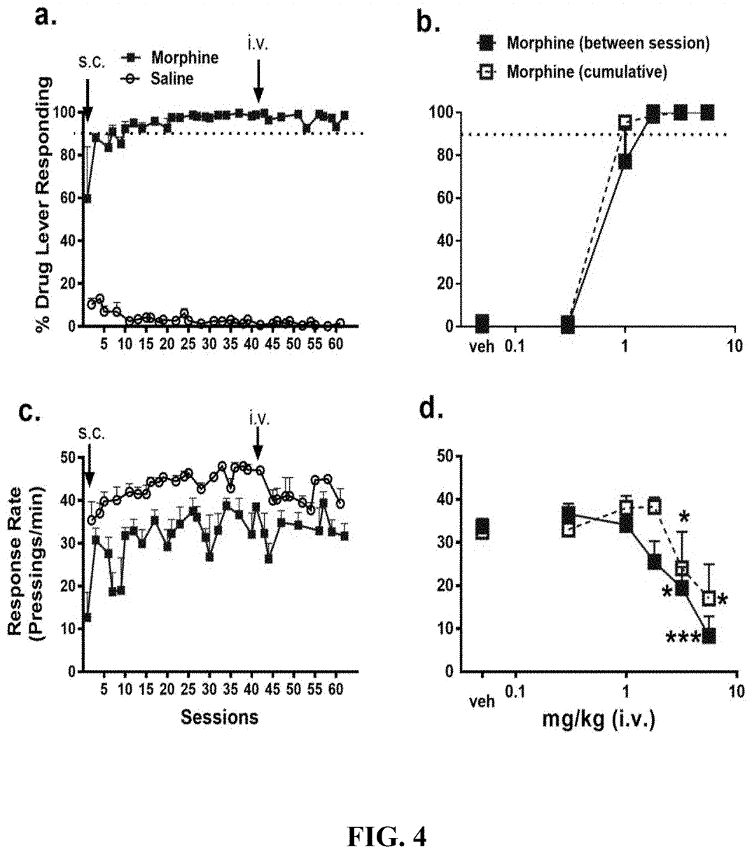

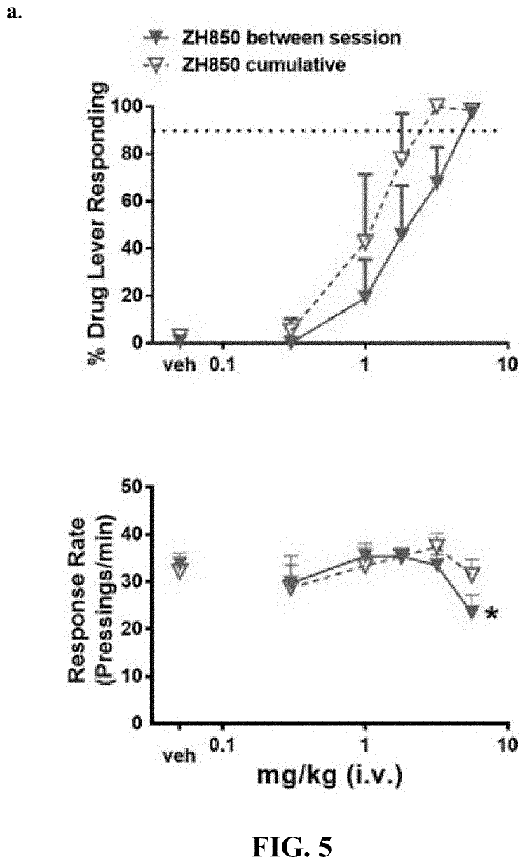

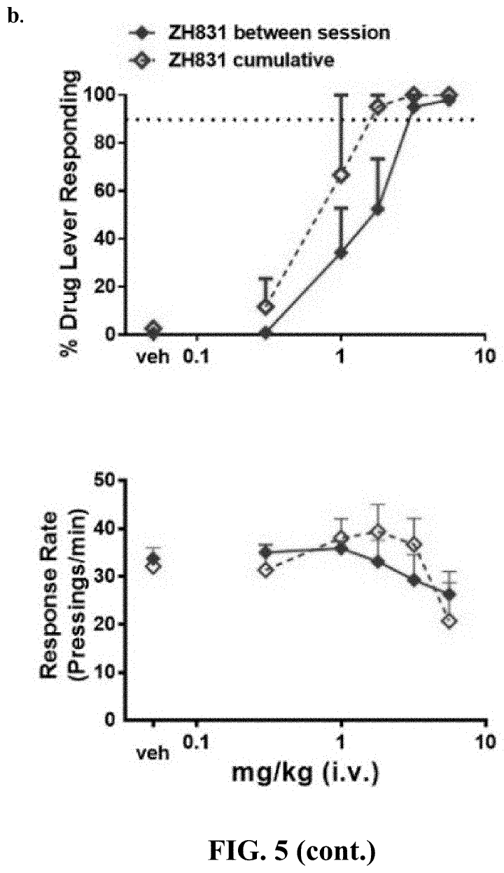

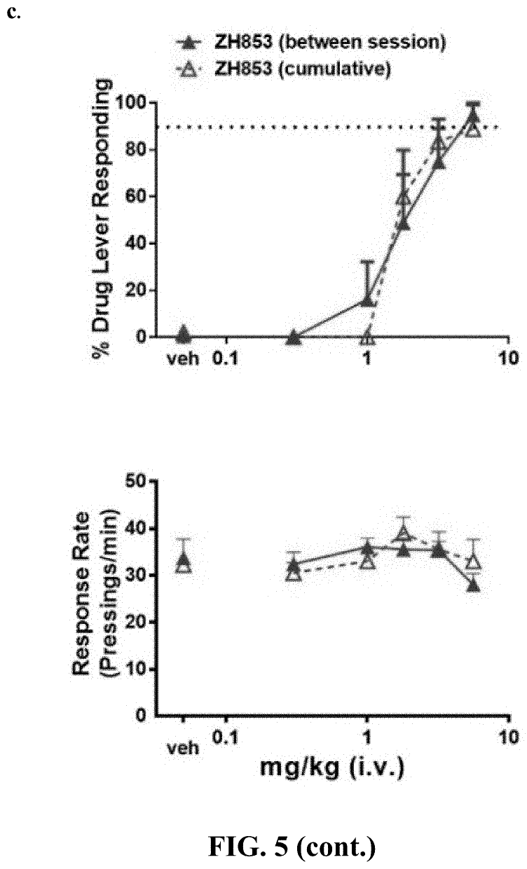

The DD effects of ZH850, ZH831, and ZH853 were studied in rats trained to discriminate s.c. and i.v. injections of morphine from vehicle (see Table 6 for procedural details). Subcutaneous morphine produced dose-dependent responding on the drug-paired lever (ED₅₀=1.247 mg/Kg) that was slightly less potent than i.v. morphine (ED₅₀=0.879 mg/Kg, data not shown), consistent with antinociceptive route of injection differences found in a previous study (South et al., 2009). After approximately 20 sessions, morphine produced>90% appropriate drug-lever responding while vehicle produced only 0.6% drug-lever responding (FIG. 4, Panel a). Substitution curves were generated for morphine (FIG. 4, Panel b), ZH850, ZH831, and ZH853 (FIG. 5) in tests in which both levers delivered food. Response rates for food were modestly impaired by the training doses of morphine (3.2 mg/Kg, s.c. and 1.8 mg/Kg, i.v.) compared to saline (FIG. 4, Panel c). ZH850, ZH831, and ZH853 fully substituted for morphine under both injection procedures, but only morphine [F (_{5, 29})=9.467, p<0.0001, FIG. 4, Panel d] and, to a lesser extent, ZH850 [F(_{5, 26})(s, =3.216, p=0.0216, FIG. 4, Panel a] reduced response rates compared to vehicle. Response rates were not impaired by ZH831 [F (_{5, 28})=1.391, p=0.2578 n.s., FIG. 4, Panel b] or ZH853 [F (_{5, 29})=2.063, p=0.0991 n.s., FIG. 4, Panel c]. ZH853 response rate impairment scores were the lowest among all compounds tested. Table 7 shows relative ED₅₀'s after bolus or within-session cumulative injections of morphine, ZH850, ZH831, or ZH853. Cumulative injections produced slightly more potent ED₅₀ values for all compounds compared to bolus injections made between sessions.

FIG. 5 illustrates discriminative stimulus and response rate effects of EM analogs in morphine-trained rats. Morphine-appropriate lever responding during test sessions in which ZH850 (a), ZH831 (b), or ZH853 (c) were administered with bolus injections made between sessions or cumulative injections made within a single session. The bottom panels show response rates for food (pressings/min) were modestly, but significantly impaired by between-session injection of ZH850, but not after cumulative injections. ZH831 and ZH853 did not impair response rates under either injection method, and fully substituted for morphine. *p<0.05 compared to vehicle. n=6.

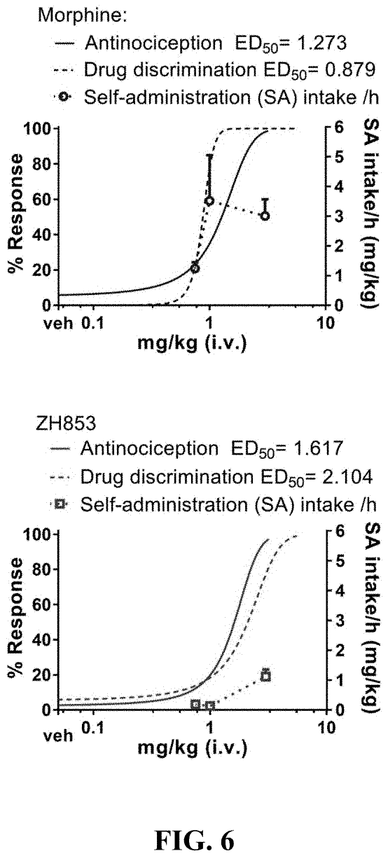

Comparing the self-administration (SA) data during 12-hour SA sessions from our previous study (Zadina et al., 2016), we found that, at a dose of 1 mg/Kg/infusion, the hourly SA intake of morphine was ˜3.5 mg/Kg/h, relatively higher than the antinociceptive ED₅₀ (1.27 mg/Kg), and the DD ED₅₀ (0.88 mg/Kg) (FIG. 6). Comparing the antinociceptive and drug discrimination dose-response curves (lefty-axis) for ZH853 to self-administration intake/h (mg/Kg, righty-axis) SA sessions, ZH853 peaked at 1.1 mg/Kg/h at the 3 mg/Kg/infusion dose, but this was relatively lower than the antinociceptive ED₅₀ and the ED₅₀ for DD. The SA infusion doses of 1 and 3 mg/Kg/infusion of ZH853 corresponded roughly with the ED₂₀ and ED₈₀ in the DD model, respectively. Similarly, the 1 mg/Kg/infusion dose corresponded to the antinociceptive ED₂₀, and the 3 mg/Kg/infusion dose produced maximal (100% MPE) antinociception in tail flick tests (FIG. 6). Thus, rats self-administered a larger amount of morphine than maximal doses producing antinociception or DD, while for ZH853, the hourly SA intake remained below the level producing antinociception and morphine-substitution effects.

TABLE 6
Training Schedule for Drug Discrimination

| | | | Stimulus Light |
|---|---|---|---|
| Phase | Schedule | Lever for Food | Manifested |
| 1a | Food training | Both levers (FR1) | Both |
| 1b | Food training | Only Left or Only | Only Correct |
| | | Right (FR10) | lever |
| 2a | Morphine (3.2 mg/kg, s.c.) | Only Morphine or | Only Correct |
| | training | Vehicle lever | lever |
| 2b | Morphine (3.2 mg/kg, s.c.) | Only Morphine or | Both |
| | training | Vehicle lever | |
| 2c | Morphine s.c. testing | Both levers | Both |
| 3a | Morphine (3.8 mg/kg, i.v.) | Only Morphine or | Both |
| | training | Vehicle lever | |
| 3b | Morphine or EM analog i.v. | Both levers | Both |
| | testing | | |

TABLE 7
Discriminative stimulus effects of EM analogs in male rats
trained to discriminate morphine injections from saline.*

| | Bolus injections | | Cumulative injections | |
|---|---|---|---|---|
| | Maximum | | Maximum | |
| | % drug- | | % drug- | |
| | appropriate | | appropriate | |
| Drug | responding | ED₅₀ | responding | ED₅₀ |
| Morphine | 100 | 0.879 (0.365) | 100 | 0.701 (0.046) |
| ZH850 | 97.2 | 2.320 (0.328) | 98.2 | 1.201 (0.170) |
| ZH831 | 98.0 | 1.631 (0.205) | 100 | 0.830 (0.131) |
| ZH853 | 94.9 | 2.104 (0.283) | 89.0 | 1.765 (2.333) |
| | Response | Response | Response | Response |
| | rate | rate | rate | rate |
| | impairment | impairment | impairment | impairment |
| | % | SEM | % | SEM |
| Morphine | 77.1 | 11.6 | 54.5 | 19.1 |
| ZH850 | 30.4 | 11.4 | −0.3 | 11.7 |
| ZH831 | 21.1 | 8.8 | 30.6 | 27.0 |
| ZH853 | 15.1 | 10.2 | 0.8 | 2.8 |

*Values represent the % maximum morphine-appropriate responding, ED₅₀ (mg/Kg, i.v. [SEM]) after bolus injections made between sessions, or cumulative injection made within a single session. Response rate impairment with the highest dose tested (5.6 mg/Kg) as a percent of vehicle responding and SEM of 5-6 rates/group.

### Example 5—Morphine, but not Z11853, Reinstates Morphine-Induced Conditioned Place Preference

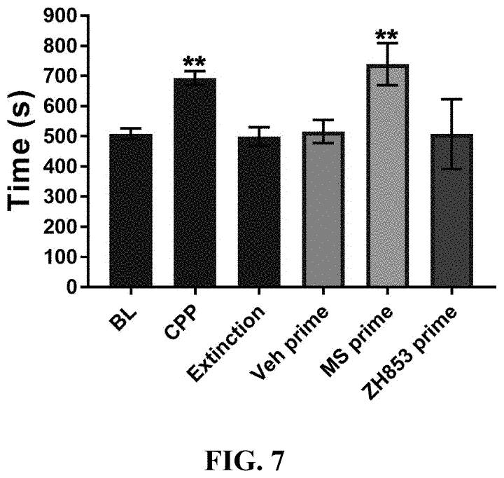

As can be seen in FIG. 7, animals that develop a preference for a standard opioid (morphine) will readily reinstate that preference after extinction in response to a single priming dose of morphine, while an equi-antinociceptive dose of ZH853 will not serve this reinforcing function.

### Example 6: Morphine, but not Z11853, Produces Naloxone-Induced Conditioned Place Aversion

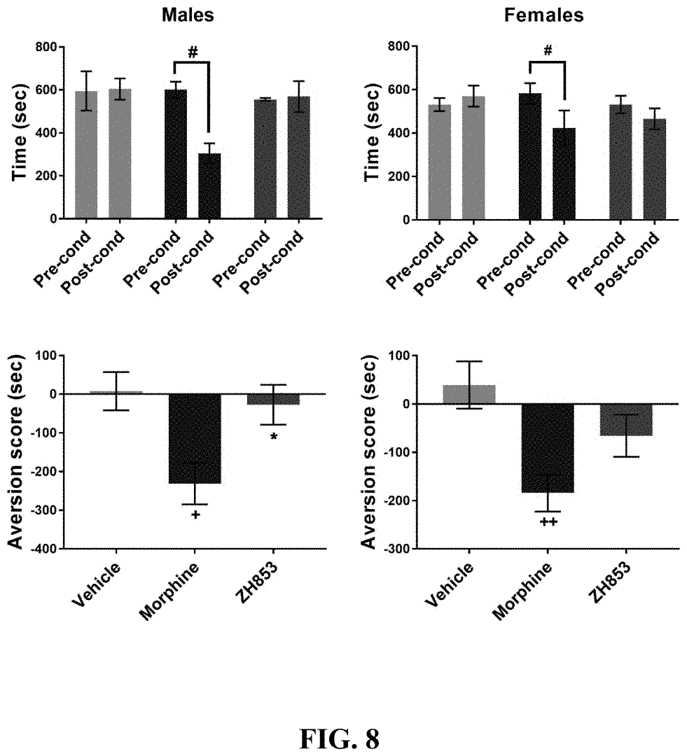

To assess whether ZH853 induces the negative affect typical of opioid withdrawal, the conditioned place aversion (CPA) test was conducted. As can be seen in FIG. 8, consistent with an aversive effect of treatment with morphine then naloxone, a decrease in time spent on the naloxone-paired side was observed for both males and females after that treatment. By contrast, ZH853+naloxone did not produce a place aversion. Lower graphs: Expression as an aversion score (post-conditioning minus pre-conditioning time in the naloxone paired chamber) shows that morphine, but not ZH853, produces aversive behavior. (#, ##, p<0.05, 0.01; +, ++=p>0.05, 0.01 vs vehicle, *=p<0.05 morphine vs ZH853. n=7-8 per group).

### Methods:

Animals: Male Sprague Dawley rats were purchased from Charles River (Wilmington, Mass.) and housed in a 12 h light/dark cycle at 22° C. with 30-70% humidity. Rats arrived at 3 months old weighing approximately 260-300 g and were housed 2 per cage until surgery. After surgery, rats were single-housed. For the hot plate study, DBA male and female mice were purchased from Charles River and housed under a 12 h light/dark cycle at 22° C. with 30-70% humidity. Mice weighed approximately 20-25 g and were housed 4-5 per cage. All experiments were approved by the Tulane Institutional Animal Care and Use Committee and conducted according to the NIH Guide for the Care and Use of Laboratory Animals.

Drugs: EM analogs were synthesized by standard solid phase methods at 1 mmol on a Rink amide resin via Fmoc chemistry with purity (>95%) and sequence identity confirmed by HPLC and MS. Analogs selected for full characterization and used here were synthesized at 2-4 g scale by American Peptide Company/Bachem (Torrance, Calif.). These included: Tyr-c[D-Lys-Trp-Phe-Glu]-NH₂ (ZH850), Tyr-c[D-Glu-Phe-Phe-Lys]-NH₂ (ZH831) and Tyr-c[D-Lys-Trp-Phe-Glu]-Gly-NH₂ (ZH853) (Zadina et al., 2016). Morphine sulfate was supplied by NIDA, beta-funaltrexamine (βFNA) and naloxone were obtained from Sigma (St. Louis, Mo.). Morphine sulfate and βFNA were dissolved in saline and ZH850, ZH831, and ZH850 were dissolved in 20% PEG-400/saline.

Intravenous catheter implantation: Rats were catheterized in the left jugular vein (Wade et al., 2015; Zadina et al., 2016). Rats were anesthetized with an isoflurane/oxygen mixture (4-5% induction, and 1.5-2.5% for the remainder of the surgery). A 1 cm area on the ventral and a 2 cm area on the dorsal side of the rat were shaved and sterilized for incision. The catheter was passed subcutaneously from the back, inserted into the left jugular vein, and secured with sutures. Wounds were sutured and dressed with antibiotic ointment and rats were given a subcutaneous injection of 0.5% lidocaine and 0.25% bupivacaine for incisional pain. All rats were allowed 5 days to recover from surgery prior to behavioral testing. Catheters were flushed every other day with 0.1 mL of streptokinase (0.134 mg/mL) to maintain catheter patency. Rats with questionable catheter patency were tested with an injection of the ultra-short acting barbiturate anesthetic, methohexital (0.1 mL of 10 mg/mL). If muscle tone was not lost within 3 seconds, the catheter was considered faulty and the rat was excluded from the analysis.

Conditioned Place Preference (CPP) and locomotor activity: Standard CPP chambers (TSE; Chesterfield, Mo.) were used to measure baseline activity and conditioning trials. Baseline activity was measured over 2 days with 4 trials per day (2 in the AM, and 2 in the PM) lasting 20 min each. Conditioning trials were conducted immediately after injection of morphine, ZH853, or vehicle and rats were restricted to distinct compartments (striped vs. gray walls) for 30 min. Doses of morphine or ZH853 (1.8, 3.2, and 5.6 mg/Kg, i.v.) were chosen based on antinociceptive % MPE levels of 70-80%, 100%, and a ¼ log dose higher, respectively (Zadina et al., 2016). Conditioning trials were conducted for 5 days and tested in an unbiased manner such that drug/environment pairings were counterbalanced for time of drug injection (AM or PM) and compartment (preferred or non-preferred) based on baseline activity. Analysis of data from our previous study using this design in a 3-day paradigm (Zadina et al., 2016) showed no difference in the effects of AM vs PM administration of ZH853 and no interaction of drug dose x time of administration on CPP scores. This indicates a lack of carry-over effects of drug between sessions. One day following the last conditioning trial, a 20 min test trial was conducted in the same manner as baseline trials in which rats were free to explore both compartments. Change in time spent exploring the drug-paired compartment (test-baseline) was used to indicate preference (or aversion) to the compartment. Approximately 20 min following the final session, rats were perfused and brain samples were taken for immunohistochemical analysis (below). Locomotor activity was assessed by infrared light beams located in the conditioning compartments and the start box. Locomotor activity was assessed during conditioning with morphine, ZH853, or vehicle immediately following injection for the entire duration of the 5 daily conditioning trials, and during test sessions in which no drug injections were made. Data were recorded in meters and an assessment of locomotor sensitization was made by subtracting the first session from the final session.

DA morphology analysis: Fifteen min following the final CPP test session, rats were anesthetized with a mixture of ketamine/xylazine (85/10 mg/Kg, i.p., respectively) and perfused intracardially with 200 mL of 0.1 mol/L phosphate buffered saline (PBS) immediately followed by 300 mL of 4% paraformaldehyde in 0.1 mol/L PBS (pH=7.4). Brains were removed and post-fixed at 4° C. in the same fixative for 18 h. After post-fixation, brains were incubated in 30% sucrose at 4° C. for 2 days and sectioned coronally on a cryostat at 40 μm at the level of the posterior VTA (Spiga et al., 2003; Chu et al., 2008). After 2 consecutive washes in PBS, sections were blocked with 5% normal donkey serum (NDS) for 1 h, and incubated with the primary antibody anti-tyrosine-hydroxylase (anti-TH, 1: 3000 Cell Signaling Technology, Danvers, Mass.) overnight at 4° C. Slices were washed twice in PBS, re-blocked for 1 h, and incubated with the secondary antibody ALEXA594 (Life Technologies, Carlsbad, Calif.), for 2 h. Sections were washed, mounted on slides with PROLONG GOLD (Life Technologies), and stored at 4° C. Posterior VTA sections were verified according to the atlas of Paxinos and Watson (2007). Images of the parabrachial pigmented area (PBP) and the paranigral area (PN) subregions of the posterior VTA were captured in z-stacks (1 μm) with at least 5 tissue slices per rat and 5-6 rats per drug group. STEREO INVESTIGATOR software (MBF Bioscience; Williston, Va.) was used to quantify soma size using the optical fractionator probe to survey a sample of neurons in each z-stack while the nucleator probe was used to measure the cross-sectional area and volume of each cell soma. The optical fractionator probe was used to quantify the number of cells in a particular section of tissue through systematic random sampling. Between 12 and 16 regions were surveyed per z-stack. While the optical fractionator probe utilized stereological techniques to select a random sample to be analyzed, the nucleator probe measured the cross-sectional area of each neuron. Therefore, the simultaneous use of these probes systematically assessed neurons in the PBP and PN regions of the VTA in each subject, and provided morphological data for these cells including surface area (μm²) and volume (μm³). Cell somas were eligible to be quantified if they were located within the counting frame and/or if the soma touched either of the nucleator frame's green borders. Neurons were ineligible if located outside the counting frame and/or if their soma touched the frame's red borders. After determining this optimum depth, the center of the soma was located and analyzed by the nucleator probe. To eliminate bias associated with tissue orientation, nucleator rays were randomly arranged between quantifications. The nucleator probe accounted for tissue thickness and the cross-sectional area to determine surface area and volume of cell somas. All images and data analysis were collected by a blinded investigator.

Hot plate test: The hot plate (HP, IITC, Woodland Hills, Calif.) antinociceptive test, which reflects a supraspinally organized complex response (Chapman et al., 1985), was used to assess the CNS penetration of ZH853. The HP apparatus was set to 55.5° C., a temperature that elicited a response after 7-9 sec. Three baseline HP latencies to rapidly lift, lick, or shake the hind paws were taken prior to drug injection. Mice were removed from the HP after a maximum of 30 sec. βFNA or vehicle was injected 24 h prior to HP testing. Mice were then injected with ZH853 (0-10 mg/Kg s.c.) and tested 30, 45, 60, 90, 120, 180, and 240 min after injection. Data were converted to maximum possible effect (% MPE) values by the following formula ([latency-baseline latency]/[30-baseline latency])*100. These values were then converted to area under the curve (AUC) for statistical analysis.

Drug Discrimination (DD): Rats were food deprived to approximately 85% of the weight of free feeding cohorts to establish operant responding for 45 mg food pellets (Bioserve, Frenchtown, N.J.) in standard operant chambers (MED Associates, St. Albans, Vt.). Rats were fed 10-20 g of standard food after daily DD sessions, and ˜3× the daily amount on weekends to maintain appropriate weight. DD consisted of 3 phases (Krivsky et al., 2006). In phase 1, rats were trained to lever press for food at a fixed ratio (FR) level 1 (i.e., 1 lever press=1 food pellet) using 2 levers with stimulus lights located above each lever and a food hopper between the levers during 120 min sessions. Responding on either lever extinguished the light and delivered a food pellet. Once the rats earned 100 food pellets in the 120 min session for 2 consecutive sessions, response requirements progressively increased to FR2, FRS, FR7, and FR9. Once >20 pellets were earned at FR9, sessions were shortened to 20 min and response requirements were set at FR10 and the left or right lever reinforced responding on alternate days. Once >20 pellets were earned on each lever at FR10, rats started phase 2 of DD training. Phase 2 consisted of pretreatment with vehicle or morphine (3.2 mg/Kg, s.c.) 30 min prior to a 20 min session and food pellets were only available on the vehicle (saline) or morphine lever, as indicated by a light above the lever. Half the rats were trained with morphine on the left lever and vehicle on the right while the other half were trained vice-versa.

The daily order of vehicle (V) or morphine (M) injections were rotated on a 4 week cycle: week 1: M, V, V, M, V; week 2, V, M, M, V, M; week 3: V, V, M, M, V; week 4: M, M, V, V, M. When >20 pellets were earned on both morphine and vehicle days, both stimulus lights were illuminated such that only the injection prompted lever responding. Only responses on the correct lever were reinforced and responses on the incorrect lever reset the response requirement on the correct lever. Criteria to move to phase 3 were as follows: >20 pellets per session, >90% injection appropriate lever responding, and <9 responses on the incorrect lever before the first food pellet reward. At this point, substitution tests of s.c. morphine were conducted in which a range of morphine doses (0, 0.3, 1, 1.8, 3.2 mg/Kg) were administered on Mondays and Thursdays, when pressings on either lever were reinforced, and training sessions were administered on the intervening days. Test session pretreatment times were the same as phase 2, with morphine, ZH850, ZH831, ZH853, or control injections made 30 min prior to a 20-min test session.

After completion of the s.c. dose response for morphine, intravenous (i.v.) catheters were implanted as described above. Training sessions continued using the i.v. route with a ¼ log lesser dose of morphine (1.8 mg/Kg), because the antinociceptive potency of i.v. morphine is slightly greater than that of the s.c. route (South et al., 2009). Two paradigms were utilized to generate substitution curves: cumulative within-session and bolus between-session injections. For the between-session procedure, substitution curves were generated for morphine, ZH850, ZH831, and ZH853 (0, 0.3, 1, 1.8, 3.2, 5.6 mg/Kg, i.v.) in a computer-randomized order on Mondays and Thursdays using the i.v. route; food reinforcement occurred after meeting the FR10 requirement on either lever.

Training sessions were administered on the intervening days to ensure accurate responding and substitution tests only occurred after rats met the criteria described above for 4 days and met the criteria on the most recent vehicle or morphine training session. During substitution tests, both levers actively delivered food at an FR10 schedule. The cumulative injection model was conducted within a single session with morphine, ZH850, ZH831, and ZH853, or vehicle (Varner et al., 2013). Doses were increased in ¼ log increments, with injections every 20 min followed 15 minutes later by DD sessions lasting 5 min each. Doses of morphine, ZH850, ZH831, or ZH853 were increased cumulatively by injecting 0.3, 0.7, 0.8, 1.4, and finally 2.4 mg/Kg to achieve doses of 0.3, 1, 1.8, 3.2, and 5.6 mg/Kg. The advantage of this procedure was that an entire dose-response was generated in one session.

Statistics: PRISM software (GraphPad, San Diego, Calif.) was used for one-way analysis of variance (ANOVA) with Newman-Keuls post-hoc comparisons to compare CPP groups, locomotor effects, hot plate, and DD response rates. Locomotor data recorded during the conditioning sessions were analyzed by 2-way ANOVA (session x drug) and data from the first session were subtracted from the final session to assess locomotor enhancement. Experimenters were blinded to treatment groups. Drug discrimination (DD) data were analyzed by non-linear regression to calculate the ED50 for morphine, ZH850, ZH831, and ZH853.

### Example 7: Alleviation of Morphine Withdrawal Symptoms

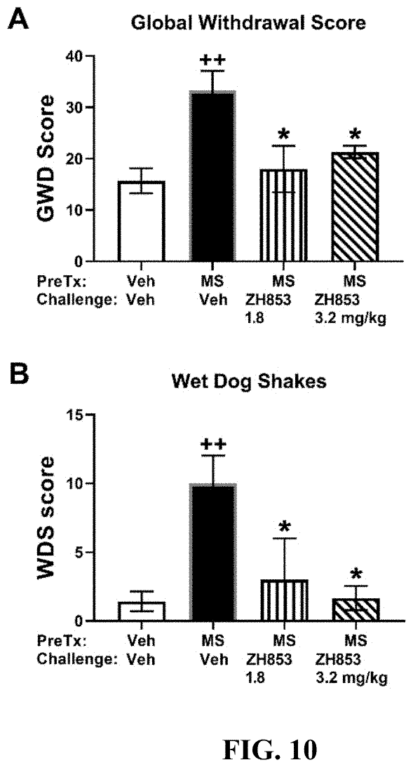

Rats were pretreated (PreTx) for 5 days with vehicle (Veh) or escalating doses of morphine sulfate (MS), then allowed 24 hr to develop spontaneous withdrawal symptoms (jumping, grooming, head shakes, wet dog shakes, and teeth chattering; collectively referred to as “global withdrawal symptoms”) The animals were then challenged with Veh or ZH853 at 1.8 or 3.2 mg/Kg and withdrawal symptoms where quantified by behavioral scores based on Ferrini et al. 2013). The results were analyzed by Analysis of Variance (ANOVA). Results are presented in FIG. 10.

Analysis of Variance of the Global Withdrawal (GWD) Scores (FIG. 10, Panel A) revealed a significant (p<0.05) effect of treatment, and post-hoc Newman-Kuels tests showed that, when challenged with Veh, animals given MS PreTx (MS-Veh) produced significantly greater withdrawal (p<0.01, ++) than Veh-PreTx+Veh challenge (Veh-Veh). By contrast, challenge with 853 (1.8 and 3.2 mg/Kg) after morphine PreTx produced significantly lower GWD scores than MS-Veh (p<0.05, *), consistent with blockade of MS-induced withdrawal.

FIG. 10, Panel B shows results for alleviation of wet dog shake (WDS), which is a component of GWD. Analysis of WDS scores revealed a significant effect of treatment (p<0.01), and a significant increase in WDS after MS PreTx+Veh challenge relative to Veh-Veh (p<0.01, ++). MS PreTx and challenge with ZH853 produced significantly lower WDS than Veh challenge for both doses of ZH853 (p<0.05, *), consistent with blockade of MS-induced withdrawal (n=7,7,3, and 3 for Veh-Veh, MS-Veh, MS-ZH853/1.8, and MS-ZH853/3.2, respectively).

### Example 8: Oxycodone, but not ZI1853, Substitution Maintains Established Oxycodon Self-Administration (SA) in Male Rats

Self-administration (SA): Self-administration tests were conducted in standard operant chambers (MED Associates, St. Albans, Vt.). Infusions were delivered through TYGON tubing threaded through metal spring leashes and attached to exterior infusion pumps. 3-hour sessions were conducted Monday-Friday during the dark cycle. At the start of each session, the house light was illuminated, and levers extended. Pressings on the active lever resulted in activation of the infusion pump and a 20-second timeout period during which the stimulus light above the active lever was illumined, and the house light was extinguished. For the first 5 sessions, rats were trained to lever press for oxycodone (0.1 mg/kg/infusion) on a FR1 reinforcement schedule. For the following 10 sessions, the dose of oxycodone was decreased to 0.05 mg/kg/infusion to increase lever responding. Inactive lever pressings were recorded but had no scheduled consequence. The criterion for successful acquisition of oxycodone SA was (1)≥15 active lever pressings in each of the last 3 sessions, and (2) a ratio of active/inactive of ≥1.5 for the last 3 sessions. Animals that did not meet criterion were excluded from subsequent phases of testing.

Maintenance of established oxycodone self-administration: Following acquisition of oxycodone self-administration, animals were split into treatment groups counterbalanced for total oxycodone intake, average number of infusions obtained in the last 3 sessions, and average number of active lever pressings over the last 3 sessions. Ten maintenance sessions were conducted in the same way as oxycodone acquisition except rats were either maintained on 0.1 mg/kg/infusion oxycodone or had drug availability substituted with equi-antinociceptive 1 mg/kg/infusion ZH853 or vehicle.

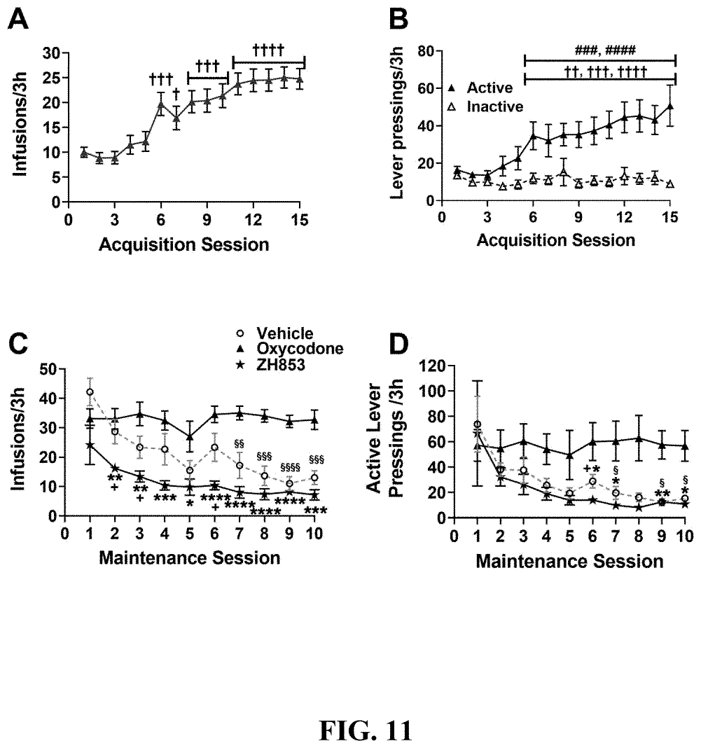

As shown in FIG. 11, after rats had acquired self-administration (SA) of oxycodone, the drug-seeking behavior was maintained if oxycodone continued to be available, as expected. However, if the available drug was switched to ZH853 or vehicle, the self-administration behavior extinguished over time. Interestingly, the rate of extinction with ZH853 availability was faster than with vehicle substitution. This is consistent with low abuse liability for ZH853, even in animals previously showing opioid-induced SA.

### Example 9: ZH853 Inhibits Oxycodone-Seeking Behavior after Forced Abstinence in Male Rats

Forced abstinence and relapse test: Following acquisition of oxycodone self-administration, rats underwent 7 days of forced abstinence from oxycodone and exposure to the testing environment in their home cages. Animals were then split into treatment groups counterbalanced for total oxycodone intake, average number of infusions obtained in the last 3 sessions, and average number of active lever pressings over the last 3 sessions. On the 8^{th} day of forced abstinence, rats were given an intravenous injection of 1.8 mg/kg ZH853, 3.2 mg/kg ZH853, or vehicle. The relapse test began 30 minutes later. The 1-hour session was conducted in the same way as oxycodone acquisition except active lever pressings resulted in activation of the pump but no drug infusion. ZH853 doses were based on previous findings (Nilges et al., 2019) that 1.8 and 3.2 mg/kg ZH853 substituted for morphine without impairing lever pressing for food.

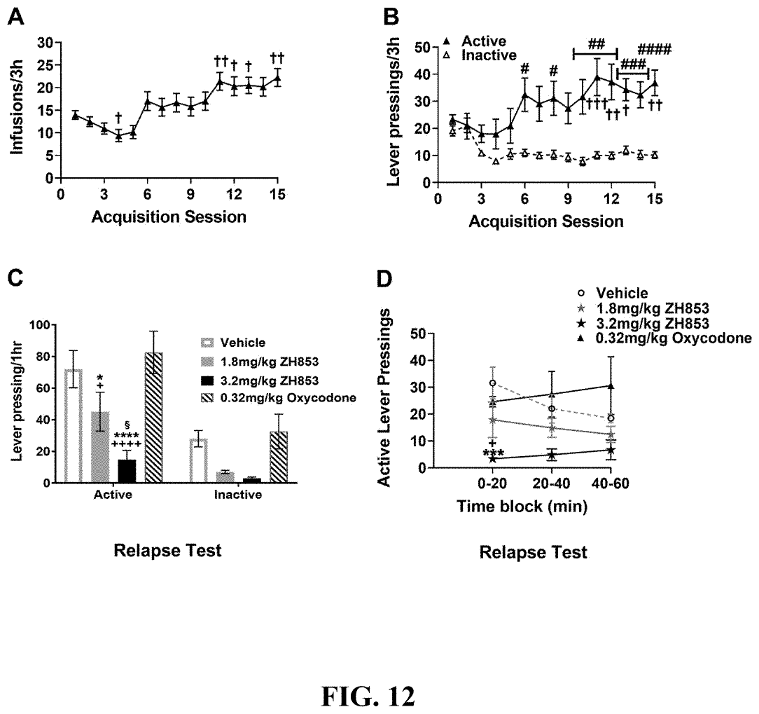

As shown in FIG. 12, rats acquired SA of oxycodone, then were subjected to 7 days of forced withdrawal. Oxycodone and vehicle pretreatment were associated with a relapse to SA behavior, but pretreatment with ZH853 dose-dependently inhibited SA behavior. This is consistent with the idea that ZH853 treatment can inhibit relapse to addictive opioids.

### Discussion

As described herein, ZH853 does not produce rewarding effects, despite CNS penetration, as reflected in SA, CPP, and LS paradigms. In addition, changes in VTA DA neurons associated with drugs of abuse like morphine were not observed with ZH853. Drug discrimination tests, however, show that ZH853, and a related EM analog (ZH831), were able to fully substitute for morphine. Since the substitution effects occurred without response rate disruption, ZH831 and ZH853 may have favorable profiles for the treatment of OUD.

Upon conditioning for 5 days with ZH853 did not induce CPP or locomotor sensitization (LS; FIG. 1) and, following this procedure, ZH853 did not reduce the size of DA cell somas (FIG. 2), whereas morphine produced CPP, LS and decreased DA cell-soma size in the VTA. The lack of response rate disruption during DD substitution and evidence that ZH853 does not produce SA, CPP, LS, or DA cell soma morphology alterations indicate a therapeutic range in which ZH853 substitutes for morphine with reduced abuse liability.

Extending a previous CPP study in which ZH853 was injected over 3 daily pairings, ZH853 did not produce CPP effects after 5 daily pairings. One explanation for this may be that locomotor sensitization (LS) did not occur during conditioning sessions with ZH853, whereas morphine induced robust LS effects in addition to CPP. Reinstatement of heroin self-administration has been associated with the expression of LS, since animals that previously self-administered heroin showed exaggerated locomotor responses upon challenge with heroin, cocaine, and amphetamine compared to controls (De Vries et al., 1999). The lack of LS induced by ZH853 may also explain why ZH853 was not self-administered (SA) in the previous study (Zadina et al., 2016), since most compounds that produce SA and CPP effects also produce LS (Robinson and Berridge, 2001). One exception to this is tramadol which does not produce LS, but does produce CPP (Tzschentke, 2004) and a low SA response rate (O'Connor and Mead, 2010) indicating that these abuse potential effects are separable. The lack of SA, LS and CPP by ZH853 is an important finding, because these effects were tested at doses that produced antinociceptive and morphine-discriminative stimulus effects.

Following CPP, DA cell soma morphology was analyzed in these rats since morphine and heroin have been reported to reduce DA cell-soma size in the VTA in post mortem human heroin users (Mazei-Robison et al., 2011), rats (Spiga et al., 2003; Russo et al., 2007; Chu et al., 2008) and mice (Mazei-Robison et al., 2011). These pVTA neurons synapse in the nucleus accumbens (NAc) and release DA at median spiny neurons that project to nucleus accumbens and prefrontal cortex regions such as the anterior cingulate cortex (Ikemoto, 2007). Post mortem studies show heroin overdose deaths are associated with reduced dopamine concentration in the nucleus accumbens (Kish et al., 2001) compared to controls. Chronic morphine produces a reward-tolerance effect that coincides with hyperexcitable DA neuron firing rates, decreased DA release in the NAc, and DA soma size reductions (Sklair-Tavron et al., 1996; Mazei-Robison et al., 2011). Daily injections of morphine (5.6 mg/Kg) were sufficient to reduce the area and volume of DA neurons in the pVTA, however ZH853 did not reduce the size of DA cell somas at any dose (FIG. 2). Interestingly, this dose of morphine (5.6 mg/Kg) did not induce significant CPP, but did shrink the size of DA neurons in the pVTA, so it is possible that tolerance to the rewarding effects of this dose occurred after the 5 daily injections. For example, a lower dose of morphine (3.2 mg/Kg) did not shrink the size of these DA neurons, however this dose produced CPP and LS. Overall, these morphological changes are associated with physiological, neurochemical, and behavioral adaptations that occur during chronic opioid usage. Therefore, the absence of DA morphology alterations after 5 daily injections of ZH853 further support the behavioral studies that indicate low abuse liability.

The hot plate antinociception induced by ZH853 suggests blood-brain barrier penetration was achieved and supports our previous BBB penetration data showing that antinociception after peripheral (i.v.) injection is significantly reduced by a centrally-administered (i.c.v.) opioid antagonist. The use of mice here and rats in earlier studies indicates the generality across species. Furthermore, it is well-known that mice typically require a larger dose than rats, so the 10-mg/Kg maximum dose of ZH853 used in the present study seems appropriate. Taken together, these studies indicate that the lack of rewarding properties of ZH853 is not explained by a lack of CNS penetration.

Although the i.v. catheter patency limited examination of naloxone sensitivity to the discriminative stimulus effects of the EM analogs, tests in 2 rats showed reversal by this antagonist (data not shown). Due to the irreversible nature of β-funaltrexamine (βFNA) this compound could not be evaluated during DD test sessions, but since βFNA blocked hot plate antinociception induced by ZH853 (FIG. 3) and receptor binding and in vivo studies show high MOR selectivity (Zadina et al., 2016), it can be concluded that ZH853 is highly MOR selective, and agonist activity at this receptor may account for both the antinociceptive and discriminative stimulus effects of ZH853.

Rats trained to discriminate morphine from vehicle injections (FIG. 4) dose-dependently pressed the morphine-trained lever when pre-injected with EM analogs (FIG. 5). While morphine, and to a lesser extent ZH850, significantly decreased response rates during DD test sessions, ZH 831 and ZH853 did not decrease response rates during substitution experiments. There are several examples of opioids that substitute for morphine in the DD model. These include heroin, buprenorphine, fentanyl, oxycodone, methadone and methadone's active metabolites (Young et al., 1992; Craft et al., 1999; Beardsley et al., 2004; Vann et al., 2009). As described herein, ZH850, ZH831, and ZH853 impaired DD response rates only 30%, 21%, and 15%, after bolus infusions, and −0.3%, 31%, and 0.8% after cumulative injections, respectively. Morphine impaired response rates 77% and 55% after bolus or cumulative injections, respectively. Therefore, ZH831 and ZH853 did not disrupt responding at doses that fully substituted for morphine, while ZH850 impaired response rates only after bolus, but not after cumulative injections.

The 5.6-mg/Kg dose of ZH853 was selected as the maximum dose in these studies due to the long antinociceptive duration (about 4.5 hours) produced by this dose and the fact that this dose of ZH853 substituted for morphine supports the rational for choosing this maximum dose. The DD effects of ZH831 and ZH853 are consistent with the lack of motor impairment produced by these analogs on the Rotarod after cumulative doses that produced maximum antinociceptive effects in the prior report (Zadina et al., 2016).

The long duration of antinociceptive effects and reduced tolerance displayed previously by the EM analogs suggest fewer subsequent doses would be required to maintain morphine-substitution effects (Flugsrud-Breckenridge et al., 2007). Overall, all EM analogs tested here produced morphine discriminative stimulus effects that coincided with less response rate disruption, an effect that could have a favorable outcome for the treatment of opioid use disorder (OUD). Given that morphine has numerous physiological effects, it is unknown what stimulus is prompting the rats to respond on the morphine-paired lever when injected with ZH853. It is possible that an antinociceptive effect, which has an ED₅₀ below that of the ED₅₀ for DD (see FIG. 6) is prompting the response. The preclinical data described herein, clearly support the concept that the DD and reinforcing effects are separable.

There are at least three potential mechanisms by which such a dissociation of analgesic and discriminative stimulus effects from rewarding effects might occur. First, pharmacokinetic (PK) factors could contribute to the lack of reinforcing effects produced by ZH853. It is well-established that drugs with slower onset and longer duration of action produce fewer reinforcing effects. Of these, the onset after i.v. injection is only marginally slower than morphine, but the duration of action is longer and could contribute to the differences.

Second, a major current focus for differential agonist effects is on biased agonism, particularly with regard to G-protein activation vs β-arrestin recruitment. Preliminary studies indicate that ZH853 is a full agonist for G-protein activation and moderately recruits β-arrestin. This does not fit current descriptions of biased agonism with regard to these two signaling processes. Most current studies of biased agonism focus on G-protein biased agonists, i.e., agonists with high G-protein efficacy and extremely low β-arrestin recruitment to approximate β-arrestin knockout effects. Favorable effects of very low or absent β-arrestin recruitment are reduced respiratory depression, enhanced analgesia, and reduced GI dysfunction. Far less attention is given, however, to the demonstration that β-arrestin knockout mice displayed an increased sensitivity to the rewarding effects of morphine that included increased CPP and striatal dopamine release compared to wild-type mice (Bohn et al., 2003).

In addition to PK factors and signaling biases at the mu receptor, a third potential mechanism is the fact that differential glial activation contributes to differences in the effects of the analogs relative to morphine-like compounds. Several endomorphin analogs do not activate glia under conditions where morphine does (Zadina et al., 2016). This correlated with reduced tolerance for the analogs. Modulation of glial cells may also play an important role in reward. Several studies have linked glial reactivity to morphine-induced reward behaviors. Morphine-induced CPP was associated with increased expression of Ibal and pp38 in the nucleus accumbens (NAc) (Zhang et al., 2012). Systemic (Hutchinson et al., 2008) and intra-accumbens (Zhang et al., 2012) minocycline blocked morphine-induced CPP, and intra-NAc injection of media from cultured astrocytes potentiated CPP for morphine (Narita et al., 2006). Morphine-induced glial changes have also been shown to contribute to reward tolerance (Taylor et al., 2016). Thus, glial activation after morphine is associated with impairment of analgesia, increases in analgesic tolerance, and facilitation of reward and reward-tolerance. The lack of glial activation by ZH853 is consistent with the relative lack of these behavioral effects compared to morphine. Thus, the presence of DD and potent analgesia in the absence of reward may be mediated, in part, by the unique glial profile of ZH853.

A key effect supporting the ability of a compound to serve as a treatment for OUD would be the ability to block the withdrawal symptoms induced by discontinuing chronic exposure to an opioid such as morphine. FIG. 10 provides preliminary evidence that ZH853 has this effect. Male Sprague-Dawley rats were pretreated with vehicle or escalating doses of morphine for 5 days as follows: 10, 20, 30, and 40 mg/Kg twice daily on days 1, 2, 3, and 4, followed by one final injection of 45 mg/Kg on day 5. At 24 after the last injection, when peak withdrawal signs are known to occur, the rats were challenged with vehicle or ZH853 (1.8 or 3.2 mg/Kg). Morphine dependence was assessed using the Global Withdrawal Score (GWD score), a summary of behavior scores typically observed in rats in opioid withdrawal, modified from Ferrini, F. et al., 2013.

Jumping, grooming, head shakes, wet dog shakes, and teeth chattering were scored (0-3) in 5-minute intervals for 30 minutes. Piloerection, paw tremors, salivation, erection/ejaculation, and diarrhea were assigned 1 point per 5-minute interval if present. As expected, animals exposed to MS for 5 days and challenged with vehicle show spontaneous withdrawal, indicated by significant increases in the GWD score relative to controls exposed vehicle for 5 days to then challenged with vehicle (FIG. 10, Panel A). These results confirm the effectiveness of the dependence-inducing protocol. By contrast, exposure of dependent rats to a challenge of ZH853 (1.8 and 3.2 mg/Kg) resulted in significantly lower GWD scores (p<0.05). Separate analysis of a key component of the global score, WDS, revealed a similar pattern where both 1.8 and 3.2 mg/Kg of ZH853 reduced withdrawal symptoms relative to MS-dependent rats challenged with saline (FIG. 10, Panel B). These results are consistent with blockade of morphine withdrawal by ZH853. Additional characteristics supporting the ability of a compound to serve as a treatment for OUD would be that it does not maintain drug use in subjects previously self-administering opioids and is able to inhibit relapse after withdrawal from opioid self-administration. FIG. 11 and FIG. 12 show that ZH853 exhibits these properties.

OUD is a difficult to manage disorder that often requires chronic daily treatment with long-acting opioid drugs that may themselves produce self-administrations and behaviorally disruptive effects. ZH831 and ZH853, in particular, did not produce reinforcing effects in SA/CPP procedures, nor did these compounds disrupt response rates at doses that substituted for morphine. The antinociceptive effects of ZH853 were blocked by βFNA, indicating MOR selectivity of this compound. While morphine reduced the area and volume of DA cell somas in the VTA, ZH853 did not produce this effect. It should be emphasized that the morphine-substitution effects of ZH853 would have predicted that this analog would produce self-administrations, however this was not the case. ZH853 was not self-administered even under 12-hour access conditions, nor did it produce CPP or LS, so the reinforcing effects of a compound can be dissociated from its morphine-discriminative stimulus effects. The low abuse liability profile and potent morphine-substitution effects of EM analogs shown here, particularly ZH853, indicate that they could significantly improve treatment for this disorder

### REFERENCES

Ator N A (2002) Relation between discriminative and reinforcing effects of midazolam, pentobarbital, chlordiazepoxide, zolpidem, and imidazenil in baboons. Psychopharmacology (Berl) 163:477-487.
Beardsley P M, Aceto M D, Cook C D, Bowman E R, Newman J L and Harris L S (2004) Discriminative stimulus, reinforcing, physical dependence, and antinociceptive effects of oxycodone in mice, rats, and rhesus monkeys. Exp Clin Psychopharmacol 12:163-172.
Bohn L M, Gainetdinov R R, Sotnikova T D, Medvedev I O, Lefkowitz R J, Dykstra L A and Caron M G (2003) Enhanced rewarding properties of morphine, but not cocaine, in β arrestin-2 knock-out mice. J Neurosci 23:10265-10273.
Bordia T, Hrachova M, Chin M, McIntosh J M and Quik M (2012) Varenicline is a potent partial agonist at α6β2* nicotinic acetylcholine receptors in rat and monkey striatum. J Pharmacol Exp Ther 342:327-334.
Chapman C R, Casey K L, Dubner R, Foley K M, Gracely R H and Reading A E (1985) Pain measurement: an overview. Pain 22:1-31.
Chu N N, Xia W, Yu P, Hu L, Zhang R and Cui C L (2008) Chronic morphine-induced neuronal morphological changes in the ventral tegmental area in rats are reversed by electroacupuncture treatment. Addict Biol 13:47-51.
corner S D, Sullivan M A and Walker E A (2005) Comparison of intravenous buprenorphine and methadone self-administration by recently detoxified heroin-dependent individuals. J Pharmacol Exp Ther 315:1320-1330.
Craft R M, Heideman L M and Bartok R E (1999) Effect of gonadectomy on discriminative stimulus effects of morphine in female versus male rats. Drug Alcohol Depend 53:95-109.
De Vries T J, Schoffelmeer A N, Binnekade R and Vanderschuren L J (1999) Dopaminergic mechanisms mediating the incentive to seek cocaine and heroin following long-term withdrawal of IV drug self-administration. Psychopharmacology (Berl) 143:254-260.
Ferrini, F. et al., 2013. Morphine hyperalgesia gated through microglia-mediated disruption of neuronal Cl-homeostasis. Nat. Neurosci. 16, 183-192.
Flugsrud-Breckenridge M R, Gevirtz C, Paul D and Gould H J, 3rd (2007) Medications of abuse in pain management. Curr Opin Anaesthesiol 20:319-324.
Hepner I J, Homewood J and Taylor A J (2002) Methadone disrupts performance on the working memory version of the Morris water task. Physiol Behav 76:41-49.
Hutchinson M R, Northcutt A L, Chao L W, Kearney J J, Zhang Y, Berkelhammer D L, Loram L C, Rozeske R R, Bland S T, Maier S F, Gleeson T T and Watkins L R (2008) Minocycline suppresses morphine-induced respiratory depression, suppresses morphine-induced reward, and enhances systemic morphine-induced analgesia. Brain Behav Immun 22:1248-1256.
Ikemoto S (2007) Dopamine reward circuitry: two projection systems from the ventral midbrain to the nucleus accumbens-olfactory tubercle complex. Brain Res Rev 56:27-78.
Jones J D, Madera G and corner S D (2014) The reinforcing and subjective effects of intravenous and intranasal buprenorphine in heroin users. Pharmacol Biochem Behav 122:299-306.
Kish S J, Kalasinsky K S, Derkach P, Schmunk G A, Guttman M, Ang L, Adams V, Furukawa Y and Haycock J W (2001) Striatal dopaminergic and serotonergic markers in human heroin users. Neuropsychopharmacology 24:561-567.
Krivsky J A, Stoffel E C, Sumner J E, Inman B C and Craft R M (2006) Role of ventral tegmental area, periaqueductal gray and parabrachial nucleus in the discriminative stimulus effects of morphine in the rat. Behav Pharmacol 17:259-270.
Lewanowitsch T, White J M and Irvine R J (2004) Use of radiotelemetry to evaluate respiratory depression produced by chronic methadone administration. Eur J Pharmacol 484:303-310.
Martin T J, Kim S A, Buechler N L, Porreca F and Eisenach J C (2007) Opioid self-administration in the nerve-injured rat: relevance of antiallodynic effects to drug consumption and effects of intrathecal analgesics. Anesthesiology 106:312-322.
Mazei-Robison M S, Koo J W, Friedman A K, Lansink C S, Robison A J, Vinish M, Krishnan V, Kim S, Siuta M A, Galli A, Niswender K D, Appasani R, Horvath M C, Neve R L, Worley P F, Snyder S H, Hurd Y L, Cheer J F, Han M H, Russo S J and Nestler E J (2011) Role for mTOR signaling and neuronal activity in morphine-induced adaptations in ventral tegmental area dopamine neurons. Neuron 72:977-990.
Mazei-Robison M S and Nestler E J (2012) Opiate-induced molecular and cellular plasticity of ventral tegmental area and locus coeruleus catecholamine neurons. Cold Spring Harb Perspect Med 2:a012070.
Meltzer L T, Moreton J E and Khazan N (1978) Electroencephalographic and behavioral tolerance and cross-tolerance to morphine and methadone in the rat. Toxicol Appl Pharmacol 45:837-844.
Mucha R F, van der Kooy D, O'Shaughnessy M and Bucenieks P (1982) Drug reinforcement studied by the use of place conditioning in rat. Brain Res 243:91-105.
Narita M, Miyatake M, Narita M, Shibasaki M, Shindo K, Nakamura A, Kuzumaki N, Nagumo Y and Suzuki T (2006) Direct evidence of astrocytic modulation in the development of rewarding effects induced by drugs of abuse. Neuropsychopharmacology 31:2476-2488.
NIDA (2017) Overdose death rates. https://www.drugabuse.gov/related-topics/trends-statistics/overdose-death-rates.
Nilges M R, Laurent M, Cable C, Arens L, Vafiades J, and Zadina J E (2019) Discriminative Stimulus and Low Abuse Liability Effects of Novel Endomorphin Analogs Suggest a Potential Treatment Indication for Opioid Use Disorder, J Pharmacol Exp Ther 370:369-379.
O'Connor E C and Mead A N (2010) Tramadol acts as a weak reinforcer in the rat self-administration model, consistent with its low abuse liability in humans. Pharmacol Biochem Behav 96:279-286.
Paxinos G and Watson C (2007) The Rat Brain in Stereotaxic Coordinates. Academic Press/Elsevier, Amsterdam.
Robinson T E and Berridge K C (2001) Incentive-sensitization and addiction. Addiction 96:103-114.
Russo S J, Bolanos C A, Theobald D E, DeCarolis N A, Renthal W, Kumar A, Winstanley C A, Renthal N E, Wiley M D, Self D W, Russell D S, Neve R L, Eisch A J and Nestler E J (2007) IRS2-Akt pathway in midbrain dopamine neurons regulates behavioral and cellular responses to opiates. Nat Neurosci 10:93-99.
Sklair-Tavron L, Shi W X, Lane S B, Harris H W, Bunney B S and Nestler E J (1996) Chronic morphine induces visible changes in the morphology of mesolimbic dopamine neurons. Proc Natl Acad Sci USA 93:11202-11207.
South S M, Edwards S R and Smith M T (2009) Antinociception versus serum concentration relationships following acute administration of intravenous morphine in male and female Sprague-Dawley rats: differences between the tail flick and hot plate nociceptive tests. Clin Exp Pharmacol Physiol 36:20-28.
Spiga S, Serra G P, Puddu M C, Foddai M and Diana M (2003) Morphine withdrawal-induced abnormalities in the VTA: confocal laser scanning microscopy. Eur J Neurosci 17:605-612.
Steinpreis R E, Rutell A L and Parrett F A (1996) Methadone produces conditioned place preference in the rat. Pharmacol Biochem Behav 54:339-341.
Swedberg M D (2016) Drug discrimination: a versatile tool for characterization of CNS safety pharmacology and potential for drug abuse. J Pharmacol Toxicol Methods 81:295-305.
Taylor A M, Castonguay A, Ghogha A, Vayssiere P, Pradhan A A, Xue L, Mehrabani S, Wu J, Levitt P, Olmstead M C, De Koninck Y, Evans C J and Cahill C M (2016) Neuroimmune regulation of GABAergic neurons within the ventral tegmental area during withdrawal from chronic morphine. Neuropsychopharmacology 41:949-959.
Tzschentke T M (2004) Reassessment of buprenorphine in conditioned place preference: temporal and pharmacological considerations. Psychopharmacology (Berl) 172:58-67.
Vann R E, Wise L E, Varvel S A, Philibin S D, Walentiny D M and Porter J H (2009) Route of administration influences substitution patterns in rats trained to discriminate methadone vs. vehicle. Drug Alcohol Depend 103:124-130.
Varner K J, Daigle K, Weed P F, Lewis P B, Mahne S E, Sankaranarayanan A and Winsauer P J (2013) Comparison of the behavioral and cardiovascular effects of mephedrone with other drugs of abuse in rats. Psychopharmacology (Berl) 225:675-685.
Wade C L, Vendruscolo L F, Schlosburg J E, Hernandez D O and Koob G F (2015) Compulsive-like responding for opioid analgesics in rats with extended access. Neuropsychopharmacology 40:421-428.
Wong W, Wallace M S, Journal of Pain, 2014, 15:25-31.
Young A M, Masaki M A and Geula C (1992) Discriminative stimulus effects of morphine: effects of training dose on agonist and antagonist effects of mu opioids. J Pharmacol Exp Ther 261:246-257.
Zadina J E, Hackler L, Ge L J and Kastin A J (1997) A potent and selective endogenous agonist for the μ-opiate receptor. Nature 386:499-502.
Zadina J E, Nilges M R, Morgenweck J, Zhang X, Hackler L and Fasold M B (2016)
Endomorphin analog analgesics with reduced abuse liability, respiratory depression, motor impairment, tolerance, and glial activation relative to morphine. Neuropharmacology 105:215-227.
Zhang X Q, Cui Y, Cui Y, Chen Y, Na X D, Chen F Y, Wei X H, Li Y Y, Liu X G and Xin W J (2012) Activation of p38 signaling in the microglia in the nucleus accumbens contributes to the acquisition and maintenance of morphine-induced conditioned place preference. Brain Behav Immun 26:318-325.
Zhang Y, Picetti R, Butelman E R, Schlussman S D, Ho A and Kreek M J (2009)
Behavioral and neurochemical changes induced by oxycodone differ between adolescent and adult mice. Neuropsychopharmacology 34:912-922.

All references, including publications, patent applications, and patents, cited herein are hereby incorporated by reference to the same extent as if each reference were individually and specifically indicated to be incorporated by reference and were set forth in its entirety herein.

What is claimed is:

1. A method for treating opioid use disorder comprising:
administering to a subject in need thereof a pharmaceutical composition comprising a cyclic peptide of Formula I or a pharmaceutically acceptable salt thereof in a pharmaceutically acceptable carrier;
wherein the peptide of Formula I is administered in place of, and as a substitute for a mu opioid receptor agonist to which the subject is addicted;
Formula I is X¹-c[X²-X³-Phe-X⁴]-X⁵;
X¹ is Tyr or 2,6-Dmt;
X² is an acidic D-amino acid or a basic D-amino acid;
X³ is Trp or Phe;
there is an amide bond between the sidechains of X² and X⁴, such that the substructure X²—X³-Phe-X⁴ constitutes a ring;
X⁵ is selected from the group consisting of NHR, Ala-NHR, Arg-NHR, Asn-NHR, Asp-NHR, Cys-NHR, Glu-NHR, Gln-NHR, Gly-NHR, His-NHR, Ile-NHR, Leu-NHR, Lys-NHR, Met-NHR, Orn-NHR, Phe-NHR, Pro-NHR, Ser-NHR, Thr-NHR, Trp-NHR, Tyr-NHR, and Val-NHR; and
R is H or an alkyl group;
provided that:
when X² is an acidic D-amino acid, X⁴ is a basic amino acid, X³ is Phe, and X⁵ is NHR; and
when X² is a basic D-amino acid, X⁴ is an acidic amino acid, and X³ is Trp.

2. The method of claim 1, wherein X² is a basic D-amino acid; X⁴ is an acidic amino acid; X³ is Trp; and X⁵ is selected from the group consisting of Ala-NHR, Arg-NHR, Asn-NHR, Asp-NHR, Cys-NHR, Glu-NHR, Gln-NHR, Gly-NHR, His-NHR, Ile-NHR, Leu-NHR, Lys-NHR, Met-NHR, Orn-NHR, Phe-NHR, Pro-NHR, Ser-NHR, Thr-NHR, Trp-NHR, Tyr-NHR, and Val-NHR.

3. The method of claim 1, wherein the mu opioid receptor agonist comprises at least one agonist selected from the group consisting of morphine, oxycodone, hydrocodone, codeine, heroin, oxymorphone, and fentanyl.

4. The method of claim 1, wherein the subject previously has been treated with at least one compound selected from the group consisting of methadone, buprenorphine, naltrexone; and suboxone.

5. The method of claim 1, wherein the pharmaceutical composition is administered intravenously.

6. The method of claim 1, wherein the pharmaceutical composition is initially administered at a dose of the peptide of Formula I that is less than the analgesic ED50 for the peptide.

7. A method for treating opioid use disorder comprising:
administering to a subject in need thereof a pharmaceutical composition comprising a cyclic peptide of Formula II or a pharmaceutically acceptable salt thereof in a pharmaceutically acceptable carrier;
wherein the peptide of Formula II is administered in place of, and as a substitute for a mu opioid receptor agonist to which the subject is addicted;
Formula II is Tyr-c[X⁶-Trp-Phe-X⁷]-X⁸—NH₂;
X⁶ is D-Lys or D-Orn;
X⁷ is Asp or Glu;
X⁸ is Gly-NH₂ or a conservative substitution therefor; and
there is an amide bond between the sidechains of X⁶ and X⁷, such that the substructure X⁶-Trp-Phe-X⁷ constitutes a ring.

8. The method of claim 7, wherein X⁶ is D-Lys.

9. The method of claim 7, wherein X⁶ is D-Orn.

10. The method of claim 7, wherein X⁷ is Glu.

11. The method of claim 7, wherein X⁷ is Asp.

12. The method of claim 7, wherein the peptide of Formula II is Tyr-c[D-Lys-Trp-Phe-Glu]-Gly-NH₂.

13. The method of claim 7, wherein the mu opioid receptor agonist comprises at least one agonist selected from the group consisting of morphine, oxycodone, hydrocodone, codeine, heroin, oxymorphone, and fentanyl.

14. The method of claim 7, wherein the subject previously has been treated with at least one compound selected from the group consisting of methadone, buprenorphine, naltrexone; and suboxone.

15. The method of claim 7, wherein the pharmaceutical composition is administered intravenously.

16. The method of claim 7, wherein the pharmaceutical composition is initially administered at a dose of the peptide of Formula II that is less than the analgesic ED50 for the peptide.

17. A method for treating opioid use disorder comprising:
administering to a subject in need thereof a pharmaceutical composition comprising a cyclic peptide of Formula III or a pharmaceutically acceptable salt thereof in a pharmaceutically acceptable carrier;
wherein the peptide of Formula III is administered in place of, and as a substitute for a mu opioid receptor agonist to which the subject is addicted;
Formula III is Tyr-c[X⁹-Phe-Phe-X¹]-NH₂;
X⁹ is D-Asp or D-Glu;
X¹⁰ is Lys or Orn; and
there is an amide bond between the sidechains of X⁹ and X¹⁰, such that the substructure X⁹-Phe-Phe-X¹⁰ constitutes a ring.

18. The method of claim 17, wherein X⁸ is D-Glu.

19. The method of claim 17, wherein X⁸ is D-Asp.

20. The method of claim 17, wherein X^{m} is Lys.

21. The method of claim 17, wherein X^{m} is Orn.

22. The method of claim 17, wherein the mu opioid receptor agonist comprises at least one agonist selected from the group consisting of morphine, oxycodone, hydrocodone, codeine, heroin, oxymorphone, and fentanyl.

23. The method of claim 17, wherein the subject previously has been treated with at least one compound selected from the group consisting of methadone, buprenorphine, naltrexone; and suboxone.

24. The method of claim 17, wherein the pharmaceutical composition is administered intravenously.

25. The method of claim 17, wherein the pharmaceutical composition is initially administered at a dose of the peptide of Formula III that is less than the analgesic ED50 for the peptide.
